SEQUENCE LISTING (228 sequences)

SEQ ID NO: 1 - DNA, 10 bp, Artificial
  CLEAR sequence
gtcgcgtgac

SEQ ID NO: 2 - DNA, 10 bp, Artificial
  CLEAR sequence
ctcacctggt

SEQ ID NO: 3 - DNA, 10 bp, Artificial
  CLEAR sequence
atcacgtggc

SEQ ID NO: 4 - DNA, 10 bp, Artificial
  CLEAR sequence
gtcacgtggc

SEQ ID NO: 5 - DNA, 10 bp, Artificial
  CLEAR sequence
agcacgtgat

SEQ ID NO: 6 - DNA, 10 bp, Artificial
  CLEAR sequence
gtcacgtgat

SEQ ID NO: 7 - DNA, 10 bp, Artificial
  CLEAR sequence
ctcaagtgtg

SEQ ID NO: 8 - DNA, 10 bp, Artificial
  CLEAR sequence
ttcaggtgcc

SEQ ID NO: 9 - DNA, 10 bp, Artificial
  CLEAR sequence
gtcaggtggc

SEQ ID NO: 10 - DNA, 10 bp, Artificial
  CLEAR sequence
gtcaggtgac

SEQ ID NO: 11 - DNA, 10 bp, Artificial
  CLEAR sequence
gtcacgttat

SEQ ID NO: 12 - DNA, 10 bp, Artificial
  CLEAR sequence
gtgacgcttc

SEQ ID NO: 13 - DNA, 10 bp, Artificial
  CLEAR sequence
gtcacgtgag

SEQ ID NO: 14 - DNA, 10 bp, Artificial
  CLEAR sequence
gtcacgtgac

SEQ ID NO: 15 - DNA, 10 bp, Artificial
  CLEAR sequence
atcagctgat

SEQ ID NO: 16 - DNA, 10 bp, Artificial
  CLEAR sequence
gtcaggtgcg

SEQ ID NO: 17 - DNA, 10 bp, Artificial
  CLEAR sequence
ttcaggtgac

SEQ ID NO: 18 - DNA, 10 bp, Artificial
  CLEAR sequence
ctcaggcgcc

SEQ ID NO: 19 - DNA, 10 bp, Artificial
  CLEAR sequence
ggcacatgac

SEQ ID NO: 20 - DNA, 10 bp, Artificial
  CLEAR sequence
gccaggtggc

SEQ ID NO: 21 - DNA, 10 bp, Artificial
  CLEAR sequence
ctcacgtagg

SEQ ID NO: 22 - DNA, 10 bp, Artificial
  CLEAR sequence
agcacgtgac

SEQ ID NO: 23 - DNA, 10 bp, Artificial
  CLEAR sequence
atcacgtgat

SEQ ID NO: 24 - DNA, 10 bp, Artificial
  CLEAR sequence
ctcacctggg

SEQ ID NO: 25 - DNA, 10 bp, Artificial
  CLEAR sequence
gccaagtgac

SEQ ID NO: 26 - DNA, 10 bp, Artificial
  CLEAR sequence
gtcagctgag

SEQ ID NO: 27 - DNA, 10 bp, Artificial
  CLEAR sequence
gccacgtgtg

SEQ ID NO: 28 - DNA, 10 bp, Artificial
  CLEAR sequence
gtcacgcggc

SEQ ID NO: 29 - DNA, 10 bp, Artificial
  CLEAR sequence
gtcatgtgag

SEQ ID NO: 30 - DNA, 10 bp, Artificial
  CLEAR sequence
gtcacgtggc

SEQ ID NO: 31 - DNA, 10 bp, Artificial
  CLEAR sequence
ttcacgtgac

SEQ ID NO: 32 - DNA, 10 bp, Artificial
  CLEAR sequence
gtcacgtggg

SEQ ID NO: 33 - DNA, 10 bp, Artificial
  CLEAR sequence
ttcacctgac

SEQ ID NO: 34 - DNA, 10 bp, Artificial
  CLEAR sequence
cccacgtgac

SEQ ID NO: 35 - DNA, 10 bp, Artificial
  CLEAR SEQUENCE
gtcagctgat

SEQ ID NO: 36 - DNA, 10 bp, Artificial
  CLEAR sequence
cccacgtgcc

SEQ ID NO: 37 - DNA, 10 bp, Artificial
  CLEAR sequence
cccagttgac

SEQ ID NO: 38 - DNA, 10 bp, Artificial
  CLEAR sequence
gtcacatgtg

SEQ ID NO: 39 - DNA, 10 bp, Artificial
  CLEAR sequence
ttcaagtgct

SEQ ID NO: 40 - DNA, 10 bp, Artificial
  CLEAR sequence
gtcaggcgaa

SEQ ID NO: 41 - DNA, 10 bp, Artificial
  CLEAR sequence
ctcaagtgat

SEQ ID NO: 42 - DNA, 10 bp, Artificial
  CLEAR sequence
ttcaggtgcc

SEQ ID NO: 43 - DNA, 10 bp, Artificial
  CLEAR sequence
gccaggtgcc

SEQ ID NO: 44 - DNA, 10 bp, Artificial
  CLEAR sequence
gtcacgtgac

SEQ ID NO: 45 - DNA, 10 bp, Artificial
  CLEAR sequence
gtcacgtgac

SEQ ID NO: 46 - DNA, 10 bp, Artificial
  CLEAR sequence
gtcatgtgac

SEQ ID NO: 47 - DNA, 10 bp, Artificial
  CLEAR sequence
gtcacgtgac

SEQ ID NO: 48 - DNA, 10 bp, Artificial
  CLEAR sequence
gtcacgcggc

SEQ ID NO: 49 - DNA, 10 bp, Artificial
  CLEAR sequence
gtcacgtggc

SEQ ID NO: 50 - DNA, 10 bp, Artificial
  CLEAR sequence
gtcatgtgac

SEQ ID NO: 51 - DNA, 10 bp, Artificial
  CLEAR sequence
atcacatgac

SEQ ID NO: 52 - DNA, 10 bp, Artificial
  CLEAR sequence
ctcacgcgag

SEQ ID NO: 53 - DNA, 10 bp, Artificial
  CLEAR sequence
ctcacgtaag

SEQ ID NO: 54 - DNA, 10 bp, Artificial
  CLEAR sequence
atcacgtgag

SEQ ID NO: 55 - DNA, 10 bp, Artificial
  CLEAR sequence
gtcatgtgag

SEQ ID NO: 56 - DNA, 10 bp, Artificial
  CLEAR sequence
gtcacgtgag

SEQ ID NO: 57 - DNA, 10 bp, Artificial
  CLEAR sequence
gtcacgcggc

SEQ ID NO: 58 - DNA, 10 bp, Artificial
  CLEAR sequence
gtcaagtgac

SEQ ID NO: 59 - DNA, 10 bp, Artificial
  CLEAR sequence
gtcacgtgac

SEQ ID NO: 60 - DNA, 10 bp, Artificial
  CLEAR sequence
ctcacgtgat

SEQ ID NO: 61 - DNA, 10 bp, Artificial
  CLEAR sequence
gtcacgcgac

SEQ ID NO: 62 - DNA, 10 bp, Artificial
  CLEAR sequence
gtcacgtgat

SEQ ID NO: 63 - DNA, 10 bp, Artificial
  CLEAR sequence
ctcacctgac

SEQ ID NO: 64 - DNA, 10 bp, Artificial
  CLEAR sequence
ctcacgtggc

SEQ ID NO: 65 - DNA, 10 bp, Artificial
  CLEAR sequence
gtcatctgac

SEQ ID NO: 66 - DNA, 10 bp, Artificial
  CLEAR sequence
gccaggtgag

SEQ ID NO: 67 - DNA, 10 bp, Artificial
  CLEAR sequence
gtcacctggc

SEQ ID NO: 68 - DNA, 10 bp, Artificial
  CLEAR sequence
gtcacatggg

SEQ ID NO: 69 - DNA, 10 bp, Artificial
  CLEAR sequence
atcagatgcc

SEQ ID NO: 70 - DNA, 10 bp, Artificial
  CLEAR sequence
gtcacctgag

SEQ ID NO: 71 - DNA, 10 bp, Artificial
  CLEAR sequence
ctcccgtgag

SEQ ID NO: 72 - DNA, 10 bp, Artificial
  CLEAR sequence
ctcagctgac

SEQ ID NO: 73 - DNA, 10 bp, Artificial
  CLEAR sequence
ccttcgtgag

SEQ ID NO: 74 - DNA, 10 bp, Artificial
  CLEAR sequence
ctcactggaa

SEQ ID NO: 75 - DNA, 10 bp, Artificial
  CLEAR sequence
atcaggttac

SEQ ID NO: 76 - DNA, 10 bp, Artificial
  CLEAR sequence
gtcagaagcg

SEQ ID NO: 77 - DNA, 10 bp, Artificial
  CLEAR sequence
gtcacgagac

SEQ ID NO: 78 - DNA, 10 bp, Artificial
  CLEAR sequence
gtcacgcgct

SEQ ID NO: 79 - DNA, 10 bp, Artificial
  CLEAR sequence
gtcagctgac

SEQ ID NO: 80 - DNA, 10 bp, Artificial
  CLEAR sequence
gtcatttgag

SEQ ID NO: 81 - DNA, 10 bp, Artificial
  CLEAR sequence
gtcacgtgac

SEQ ID NO: 82 - DNA, 10 bp, Artificial
  CLEAR sequence
gtcatgtgac

SEQ ID NO: 83 - DNA, 10 bp, Artificial
  CLEAR sequence
ggcaggtgag

SEQ ID NO: 84 - DNA, 10 bp, Artificial
  CLEAR sequence
gtcacgtgat

SEQ ID NO: 85 - DNA, 10 bp, Artificial
  CLEAR sequence
cgcacgtgac

SEQ ID NO: 86 - DNA, 10 bp, Artificial
  CLEAR sequence
atcagctgtc

SEQ ID NO: 87 - DNA, 10 bp, Artificial
  CLEAR sequence
gtcagccgac

SEQ ID NO: 88 - DNA, 10 bp, Artificial
  CLEAR sequence
aacacgtgac

SEQ ID NO: 89 - DNA, 10 bp, Artificial
  CLEAR sequence
gtcacgtgca

SEQ ID NO: 90 - DNA, 10 bp, Artificial
  CLEAR sequence
gtcatgtgac

SEQ ID NO: 91 - DNA, 10 bp, Artificial
  CLEAR sequence
atcacgtgct

SEQ ID NO: 92 - DNA, 10 bp, Artificial
  CLEAR sequence
ctcatgtgat

SEQ ID NO: 93 - DNA, 10 bp, Artificial
  CLEAR sequence
gtcacatgac

SEQ ID NO: 94 - DNA, 10 bp, Artificial
  CLEAR sequence
gtcacatgag

SEQ ID NO: 95 - DNA, 10 bp, Artificial
  CLEAR sequence
tcaactgccc

SEQ ID NO: 96 - DNA, 10 bp, Artificial
  CLEAR sequence
cccatgtgac

SEQ ID NO: 97 - DNA, 10 bp, Artificial
  CLEAR sequence
ctcacgtgcc

SEQ ID NO: 98 - DNA, 10 bp, Artificial
  CLEAR sequence
gtcacgtggg

SEQ ID NO: 99 - DNA, 10 bp, Artificial
  CLEAR sequence
gtcacgtgcc

SEQ ID NO: 100 - DNA, 10 bp, Artificial
  CLEAR sequence
gtcacgtgcc

SEQ ID NO: 101 - DNA, 10 bp, Artificial
  CLEAR sequence
gtcacgtgtc

SEQ ID NO: 102 - DNA, 10 bp, Artificial
  CLEAR sequence
atcacgtgac

SEQ ID NO: 103 - DNA, 10 bp, Artificial
  CLEAR sequence
ctcacgtgac

SEQ ID NO: 104 - DNA, 10 bp, Artificial
  CLEAR sequence
atcaggtgag

SEQ ID NO: 105 - DNA, 10 bp, Artificial
  CLEAR sequence
ctcagctgtg

SEQ ID NO: 106 - DNA, 10 bp, Artificial
  CLEAR sequence
gtcgcctgac

SEQ ID NO: 107 - DNA, 10 bp, Artificial
  CLEAR sequence
gtcttgtgac

SEQ ID NO: 108 - DNA, 10 bp, Artificial
  CLEAR sequence
atcagctgac

SEQ ID NO: 109 - DNA, 10 bp, Artificial
  CLEAR sequence
gtcacgtgac

SEQ ID NO: 110 - DNA, 10 bp, Artificial
  CLEAR consensus sequence
gtcacgtgca

SEQ ID NO: 111 - DNA, 10 bp, Artificial
  CLEAR consensus sequence
gtcacgtgca

SEQ ID NO: 112 - DNA, 68 bp, Artificial
  4x CLEAR consensus sequence
ccgggtcacgtgaccccagggtcacgtgaccctgcgggtcacgtgaccctgcgggtcacgtgaccccc

SEQ ID NO: 113 - DNA, 68 bp, Artificial
  4x control sequence
ccgggaatcgtgaccccagggaatcgtgaccctgcgggaatcgtgaccctgcgggaatcgtgaccccc

SEQ ID NO: 114 - DNA, 22 bp, Artificial
  synthetic primer
ccagaagcgagagctcacagat

SEQ ID NO: 115 - DNA, 22 bp, Artificial
  synthetic primer
tgtgattgtctttcttctgccg

SEQ ID NO: 116 - DNA, 21 bp, Artificial
  synthetic primer
agagctttgcagagcgttcag

SEQ ID NO: 117 - DNA, 21 bp, Artificial
  synthetic primer
atacgcatggtctcaggtcca

SEQ ID NO: 118 - DNA, 21 bp, Artificial
  synthetic primer
atcagtgaaggaagcccatcc

SEQ ID NO: 119 - DNA, 21 bp, Artificial
  synthetic primer
acacggtgaagagtccacgaa

SEQ ID NO: 120 - DNA, 22 bp, Artificial
  synthetic primer
cattgtgatgagcgtgttctgg

SEQ ID NO: 121 - DNA, 22 bp, Artificial
  synthetic primer
aactccccggttaggaccctta

SEQ ID NO: 122 - DNA, 21 bp, Artificial
  synthetic primer
ggaagtgtcagatgatcccca

SEQ ID NO: 123 - DNA, 20 bp, Artificial
  synthetic primer
ccgtttgcctcgtggataat

SEQ ID NO: 124 - DNA, 21 bp, Artificial
  synthetic primer
tgatctccacgttcaccctga

SEQ ID NO: 125 - DNA, 21 bp, Artificial
  synthetic primer
tctccgagtcaaaccttccga

SEQ ID NO: 126 - DNA, 21 bp, Artificial
  synthetic primer
caggctttggtcttctctcca

SEQ ID NO: 127 - DNA, 20 bp, Artificial
  synthetic primer
tcacgcattccaggtctttg

SEQ ID NO: 128 - DNA, 21 bp, Artificial
  synthetic primer
agtggagaatggcacacccta

SEQ ID NO: 129 - DNA, 20 bp, Artificial
  synthetic primer
aagaagccattgtcacccca

SEQ ID NO: 130 - DNA, 21 bp, Artificial
  synthetic primer
aactgctggacatcgcttgct

SEQ ID NO: 131 - DNA, 22 bp, Artificial
  synthetic primer
cattcttcacgtaggtgctgga

SEQ ID NO: 132 - DNA, 22 bp, Artificial
  synthetic primer
acagaggaggagttccgcacta

SEQ ID NO: 133 - DNA, 21 bp, Artificial
  synthetic primer
gcttgcttcatcttgttgcca

SEQ ID NO: 134 - DNA, 19 bp, Artificial
  synthetic primer
ttgtcggcaagtggcatct

SEQ ID NO: 135 - DNA, 21 bp, Artificial
  synthetic primer
ccaaaccactcatcaaatccg

SEQ ID NO: 136 - DNA, 20 bp, Artificial
  synthetic primer
tgggtacccggatgatgtta

SEQ ID NO: 137 - DNA, 20 bp, Artificial
  synthetic primer
agatgctgctgctctcaaca

SEQ ID NO: 138 - DNA, 21 bp, Artificial
  synthetic primer
agccagattcctgcatcagtg

SEQ ID NO: 139 - DNA, 21 bp, Artificial
  synthetic primer
ataacctgcatccttccagcc

SEQ ID NO: 140 - DNA, 21 bp, Artificial
  synthetic primer
cccattttgagaggtgccagt

SEQ ID NO: 141 - DNA, 21 bp, Artificial
  synthetic primer
tgacgttacggccttctcctt

SEQ ID NO: 142 - DNA, 21 bp, Artificial
  synthetic primer
caaccaacacattcttctcca

SEQ ID NO: 143 - DNA, 20 bp, Artificial
  synthetic primer
cgctatcgtgacctgctttt

SEQ ID NO: 144 - DNA, 21 bp, Artificial
  synthetic primer
acgttacagcgtccagctcat

SEQ ID NO: 145 - DNA, 19 bp, Artificial
  synthetic primer
tctttggagctcgcattgg

SEQ ID NO: 146 - DNA, 22 bp, Artificial
  synthetic primer
ttgctctctgccagcggtacta

SEQ ID NO: 147 - DNA, 22 bp, Artificial
  synthetic primer
gcagtcagtaaccaccatcgga

SEQ ID NO: 148 - DNA, 20 bp, Artificial
  synthetic primer
cagaaggaaggagcaggagt

SEQ ID NO: 149 - DNA, 19 bp, Artificial
  synthetic primer
atgttcccgaggctgtcac

SEQ ID NO: 150 - DNA, 22 bp, Artificial
  synthetic primer
cagcacatccagagttccgagt

SEQ ID NO: 151 - DNA, 21 bp, Artificial
  synthetic primer
tgtctctttccgccatgaggt

SEQ ID NO: 152 - DNA, 21 bp, Artificial
  synthetic primer
gccaacagtgaaatcccttcc

SEQ ID NO: 153 - DNA, 21 bp, Artificial
  synthetic primer
tcagtggcattgtccttcagc

SEQ ID NO: 154 - DNA, 21 bp, Artificial
  synthetic primer
gatctcccctgttgattcggt

SEQ ID NO: 155 - DNA, 21 bp, Artificial
  synthetic primer
agccccttatttacggcattc

SEQ ID NO: 156 - DNA, 21 bp, Artificial
  synthetic primer
tgaccggcctttcttcctcta

SEQ ID NO: 157 - DNA, 21 bp, Artificial
  synthetic primer
gctctctccgttgccaaactt

SEQ ID NO: 158 - DNA, 22 bp, Artificial
  synthetic primer
gttcgatgcccctgtaactgtc

SEQ ID NO: 159 - DNA, 22 bp, Artificial
  synthetic primer
cccaggttgatgattcttttgc

SEQ ID NO: 160 - DNA, 22 bp, Artificial
  synthetic primer
gatcccagctctcctcaatacg

SEQ ID NO: 161 - DNA, 19 bp, Artificial
  synthetic primer
gccatttttgcaccgtgtg

SEQ ID NO: 162 - DNA, 20 bp, Artificial
  synthetic primer
tgcaccaccaactgcttagc

SEQ ID NO: 163 - DNA, 21 bp, Artificial
  synthetic primer
ggcatggactgtggtcatgag

SEQ ID NO: 164 - DNA, 21 bp, Artificial
  synthetic primer
tgacactggcaaaacaatgca

SEQ ID NO: 165 - DNA, 21 bp, Artificial
  synthetic primer
ggtccttttcaccagcaagct

SEQ ID NO: 166 - DNA, 20 bp, Artificial
  synthetic primer
aggaaacggttgcagaaagg

SEQ ID NO: 167 - DNA, 20 bp, Artificial
  synthetic primer
gtcttgcggagtgggaatgt

SEQ ID NO: 168 - DNA, 22 bp, Artificial
  synthetic primer
actcaccgtggttgttggtaga

SEQ ID NO: 169 - DNA, 22 bp, Artificial
  synthetic primer
tcgattggtggactctggataa

SEQ ID NO: 170 - DNA, 21 bp, Artificial
  synthetic primer
gtgatggacgaggccttattg

SEQ ID NO: 171 - DNA, 21 bp, Artificial
  synthetic primer
tgcacataaatcccaccatgc

SEQ ID NO: 172 - DNA, 19 bp, Artificial
  synthetic primer
cccccatcgatcccaataa

SEQ ID NO: 173 - DNA, 22 bp, Artificial
  synthetic primer
ccctggtgaggtacatgttgaa

SEQ ID NO: 174 - DNA, 22 bp, Artificial
  synthetic primer
tccaagctactcaagctgccag

SEQ ID NO: 175 - DNA, 21 bp, Artificial
  synthetic primer
ccagcccggattatgttgtct

SEQ ID NO: 176 - DNA, 22 bp, Artificial
  synthetic primer
cctctaaaacccgtccaaaaca

SEQ ID NO: 177 - DNA, 22 bp, Artificial
  synthetic primer
tcggtagaagtagcaggtggaa

SEQ ID NO: 178 - DNA, 21 bp, Artificial
  synthetic primer
tgctgttgactgcagagctgt

SEQ ID NO: 179 - DNA, 22 bp, Artificial
  synthetic primer
cctagcatgagtgatctcccct

SEQ ID NO: 180 - DNA, 21 bp, Artificial
  synthetic primer
atgtggaagtggcttcaggag

SEQ ID NO: 181 - DNA, 21 bp, Artificial
  synthetic primer
gggacttcttctgggaattgt

SEQ ID NO: 182 - DNA, 22 bp, Artificial
  synthetic primer
gtcaggttgctggtcaaattcc

SEQ ID NO: 183 - DNA, 22 bp, Artificial
  synthetic primer
caacgtccccagagtctttgtc

SEQ ID NO: 184 - DNA, 19 bp, Artificial
  synthetic primer
tcgggaatccagttgtccg

SEQ ID NO: 185 - DNA, 19 bp, Artificial
  synthetic primer
gccgcacaggtagaaggaa

SEQ ID NO: 186 - DNA, 19 bp, Artificial
  synthetic primer
cgttgcaggtcacatggtc

SEQ ID NO: 187 - DNA, 18 bp, Artificial
  synthetic primer
ggctgcccccgtgtttgt

SEQ ID NO: 188 - DNA, 19 bp, Artificial
  synthetic primer
ccgtagggaccaaagaagg

SEQ ID NO: 189 - DNA, 22 bp, Artificial
  synthetic primer
tggaagtcatgtgtacgagtca

SEQ ID NO: 190 - DNA, 19 bp, Artificial
  synthetic primer
gcgtcatcccggctataag

SEQ ID NO: 191 - DNA, 19 bp, Artificial
  synthetic primer
tgaggcttcacctgacgag

SEQ ID NO: 192 - DNA, 22 bp, Artificial
  synthetic primer
aagcacgtgatagaggtcagtg

SEQ ID NO: 193 - DNA, 18 bp, Artificial
  synthetic primer
cctgcgcgttctcttgtt

SEQ ID NO: 194 - DNA, 20 bp, Artificial
  synthetic primer
tgtaacagatgagaggaagc

SEQ ID NO: 195 - DNA, 20 bp, Artificial
  synthetic primer
acacaggaagtgaggcaatc

SEQ ID NO: 196 - DNA, 19 bp, Artificial
  synthetic primer
tagcgagacggtagacgac

SEQ ID NO: 197 - DNA, 19 bp, Artificial
  synthetic primer
acccgccctatttccatac

SEQ ID NO: 198 - DNA, 18 bp, Artificial
  synthetic primer
atcgcgcctagggagaaa

SEQ ID NO: 199 - DNA, 20 bp, Artificial
  synthetic primer
aataaaaagccgtgccttga

SEQ ID NO: 200 - DNA, 18 bp, Artificial
  synthetic primer
gtgaaagggcagggtgtg

SEQ ID NO: 201 - DNA, 19 bp, Artificial
  synthetic primer
cgaatcacgtgaccagagg

SEQ ID NO: 202 - DNA, 19 bp, Artificial
  synthetic primer
cttcgagatgctgcgtgat

SEQ ID NO: 203 - DNA, 21 bp, Artificial
  synthetic primer
tcccggactctaattggtctt

SEQ ID NO: 204 - DNA, 17 bp, Artificial
  synthetic primer
aggggctctgggctacc

SEQ ID NO: 205 - DNA, 17 bp, Artificial
  synthetic primer
gcccgccgctgtcactg

SEQ ID NO: 206 - DNA, 20 bp, Artificial
  synthetic primer
ttggggcagggcagatttat

SEQ ID NO: 207 - DNA, 20 bp, Artificial
  synthetic primer
caggaggaagagggcgtaca

SEQ ID NO: 208 - DNA, 17 bp, Artificial
  synthetic primer
ccgtccgcctccgtcac

SEQ ID NO: 209 - DNA, 18 bp, Artificial
  synthetic primer
ggcagcagcagcaaccac

SEQ ID NO: 210 - DNA, 20 bp, Artificial
  synthetic primer
tcccaatagcttgcagaacc

SEQ ID NO: 211 - DNA, 19 bp, Artificial
  synthetic primer
tgtcacatgacctgccaga

SEQ ID NO: 212 - DNA, 21 bp, Artificial
  synthetic primer
agaggggtagtggtggtggaa

SEQ ID NO: 213 - DNA, 20 bp, Artificial
  synthetic primer
caggcttggagtcccattct

SEQ ID NO: 214 - DNA, 18 bp, Artificial
  synthetic primer
cacaggcacccacacaaa

SEQ ID NO: 215 - DNA, 18 bp, Artificial
  synthetic primer
acgcagctggtcagcaat

SEQ ID NO: 216 - DNA, 24 bp, Artificial
  synthetic primer
acaaggaatggaaacaaagatacc

SEQ ID NO: 217 - DNA, 20 bp, Artificial
  synthetic primer
gatgcgtcttccctttttcc

SEQ ID NO: 218 - DNA, 19 bp, Artificial
  synthetic primer
atgcagcgatcagtggcgt

SEQ ID NO: 219 - DNA, 22 bp, Artificial
  synthetic primer
tccagcttcttgtcaccacctc

SEQ ID NO: 220 - DNA, 20 bp, Artificial
  synthetic primer
gccttgactcgcacttttgt

SEQ ID NO: 221 - DNA, 20 bp, Artificial
  synthetic primer
taggcgccatcgattttaag

SEQ ID NO: 222 - DNA, 22 bp, Artificial
  synthetic primer
gccacaggtagtgcaaggtctt

SEQ ID NO: 223 - DNA, 18 bp, Artificial
  synthetic primer
ttcatggcggccgtaaac

SEQ ID NO: 224 - DNA, 20 bp, Artificial
  synthetic primer
cctctcacaccctcacttcc

SEQ ID NO: 225 - DNA, 20 bp, Artificial
  synthetic primer
tctagatgacggacgacgtg

SEQ ID NO: 226 - DNA, 20 bp, Artificial
  synthetic primer
gccagctttgccagtcttac

SEQ ID NO: 227 - DNA, 18 bp, Artificial
  synthetic primer
cgagtcgcgcaagaagat

SEQ ID NO: 228 - protein, 482 aa, Artificial
  TFEB sequence + NLS sequence
MASRIGLRMQLMREQAQQEEQRERMQQQAVMHYMQQQQQQQQQQLGGPPTPAINTPVHFQSPPPVPGEVLKVQSYLENPTSYHLQQSQHQKVREYLSETYGNKFAAHISPAQGSPKPPPAASPGVRAGHVLSSSAGNSAPNSPMAMLHIGSNPERELDDVIDNIMRLDDVLGYINPEMQMPNTLPLSSSHLNVYSSDPQVTASLVGVTSSSCPADLTQKRELTDAESRALAKERQKKDNHNLIERRRRFNINDRIKELGMLIPKANDLDVRWNKGTILKASVDYIRRMQKDLQKSRELENHSRRLEMTNKQLWLRIQELEMQARVHGLPTTSPSGMNMAELAQQVVKQELPSEEGPGEALMLGAEVPDPEPLPALPPQAPLPLPTQPPSPFHHLDFSHSLSFGGREDEGPPGYPEPLAPGHGSPFPSLSKKDLDLMLLDDSLLPLASDPLLSTMSPEASKASSRRSSFSMEEGDVLPKKKRK

(19) United States
(12) Patent Application Publication
(10) Pub. No.: US 2012/0040451 A1
(43) Pub. Date: Feb. 16, 2012

(54) MOLECULES ABLE TO MODULATE THE EXPRESSION OF AT LEAST A GENE INVOLVED IN DEGRADATIVE PATHWAYS AND USES THEREOF

(75) Inventors: Andrea Ballabio, Napoli (IT); Marco Sardiello, Napoli (IT)

(73) Assignee: FONDAZIONE TELETHON, Roma (IT)

(21) Appl. No.: 13/148,737

(22) PCT Filed: Feb. 11, 2010
(86) PCT No.: PCT/EP2010/051705

§ 371 Date: Oct. 13, 2011

Related U.S. Application Data
(60) Provisional application No. 61/185,726, filed on Jun. 10, 2009.

(30) Foreign Application Priority Data
Feb. 13, 2009 (EP) 09152778.8

(51) Int. Cl. C07K 14/435; C12N 15/12; C12N 15/63; C07H 21/02

(52) U.S. Cl. 435/320.1; 530/350; 536/23.1; 536/24.5; 536/23.5

(57) ABSTRACT

A molecule being able to modulate the expression of at least a gene involved in degradative pathways so to enhance the cellular degradative pathways and prevent or antagonize the accumulation of toxic compounds in a cell and acting on a CLEAR element. Preferred molecules are: the TFEB protein, synthetic or biotechnological functional derivative thereof; chimeric molecule comprising the TFEB protein, synthetic or biotechnological functional derivative thereof; modulator of the TFEB protein activity and/or expression level. The molecule may be used in the treatment of neurodegenerative and/or lysosomal storage disorders.

### FIELD OF THE INVENTION

The invention refers to molecules able to modulate the expression of at least a gene involved in degradative pathways so to enhance the cellular degradative pathways and prevent or antagonize the accumulation of toxic compounds in a cell.

### BACKGROUND OF THE INVENTION

Lysosomes are specialized to degrade macromolecules received from the secretory, endocytic, autophagic and phagocytic pathways (1). Lysosomal storage disorders and neurodegenerative diseases such as Alzheimer's, Parkinson's, and Huntington's share as a common feature the progressive accumulation of undigested macromolecules within the cell, either proteins that tend to form pathogenic aggregates, or intermediates of the cellular catabolism. This ultimately results in cellular dysfunction and clinical manifestations with variable association of visceral (hepatosplenomegaly), skeletal (joint limitation, bone disease and deformities), hematologic (anemia, lymphocyte vacuolization and inclusions), and, most importantly, neurological involvement, with often irreversible damage and invalidating or fatal consequences. Since all of these disorders share a reduced digestive capability of the cell, it would be of great medical interest to identify molecules able to act as general enhancers of degradative pathways.

Lysosomes are organelles central to degradation and recycling processes in animal cells. Whether lysosomal activity is coordinated to respond to cellular needs remains unclear. We found that most lysosomal genes exhibit coordinated transcriptional behavior and are regulated by the transcription factor TFEB. Under aberrant lysosomal storage conditions TFEB translocated from the cytoplasm to the nucleus, resulting in the activation of its target genes. TFEB overexpression in cultured cells induced lysosomal biogenesis and increased the degradation of complex molecules, such as glycosaminoglycans (GAGs) and the pathogenic protein causing Huntington disease. Thus, a genetic program controls lysosomal biogenesis and function, providing a potential therapeutic target to enhance cellular clearing in lysosomal storage disorders and neurodegenerative diseases.

Prior art reports the description of a system to increase the activity of some cathepsins following the inhibition of the lysosomal system; however, these results are rather partial, controversial, and the molecular mechanism has not been analyzed into details. In the published literature there are no papers that reveal the presence of a lysosomal gene network or that identify TFEB as a possible modulator of the lysosomal activity.

### DESCRIPTION OF THE INVENTION

The authors of the invention identified a gene network that comprises the genes encoding lysosomal proteins of critical importance for the degradation of toxic compounds. These proteins are involved, directly or indirectly, in a high number of human diseases. The regulatory element responsible for the modulation of these genes has been identified in their promoter sequences. Such regulatory element, which authors called CLEAR, represents itself a target for the modulation—and therefore the enhancement—of the production of the lysosomal proteins responsible for the degradation of toxic compounds. Finally, a transcription factor, called TFEB, (NCBI GeneID=7942; nt=NM_{—}007162.1, protein=NP_{—}009093.1 (aa. 1-476 of Seq Id No. 228) and variants thereof) has been identified as a protein able to bind to the CLEAR element and to modulate the expression of target genes. Authors demonstrated that the lysosomal activity can be modulated by increasing or decreasing the amount of TFEB. In particular, the lysosomal enhancement resulting from the increase in TFEB levels is able to clear the cell from the toxic protein responsible for the neurodegenerative Huntington's disease.

The enhancement of the cellular degradative pathways by the activation of the lysosomal system may be advantageously used for the therapy of lysosomal storage disorders and of neurodegenerative diseases.

Such treatment may be performed by using:

1) TFEB or synthetic or biotechnological derivatives thereof, as peptide fragments, chimeric peptides etc., acting directly on the CLEAR element, responsible for the modulation of the expression of lysosomal genes and other genes involved in degradative pathways, in order to enhance the cellular degradative pathways and prevent or antagonize the accumulation of toxic compounds; and/or
2) molecules, as peptides, microRNAs, microRNA inhibitors, or any other chemicals, able to act directly or indirectly on the TFEB protein or on its amount; and/or
3) vectors for gene therapy containing TFEB, microRNAs, microRNA inhibitors, or other genes able to modulate the CLEAR regulatory network, in order to enhance the cellular degradative pathways.

CA 2525255 A1 describes the use of TFEB for cancer treatment and for modulating cell proliferation or differentiation.

WO 2007/070856 claims the use of TFEB for treating immune dysfunction. The document discloses the suppression of CD40L expression by blocking TFEB via interfering RNA molecules; moreover the document discloses the suppression of TFEB by TFEB-dimers. None of the above relates to the enhancement of TFEB amount/activity to target genes. Esumi Noriko et al., The Journal of Biological Chemistry 1997, 282, 3, 1838-1850 discloses effects of siRNA on TFEB, which correlates with the expression of VMD2. The activation of degradative pathways via the TFEB/CLEAR network is not disclosed nor suggested in the document.

US2005/255450 discloses a method for screening candidate agents to identify lead compounds for the development of therapeutic agents for treatment of neurodegenerative diseases. The document discloses experiments with yeast cells, that identified several modificators of the clearance of neurotoxic peptides, suggesting that some putative human orthologs of yeast genes should act in the same way. A possible link between TFEB expression and clearance of neurotoxic peptides, in a diagnostic perspective, is suggested, with no data. As a matter of fact HMS1, the described yeast protein, is not the yeast ortholog of TFEB.

Finally, the CLEAR regulatory element—allowing the lysosomal system modulation—is not disclosed in any prior art documents.

Technologies able to enhance the lysosomal activity have not been described so far. Authors defined molecular events involved in the modulation of the lysosomal system through the regulatory element CLEAR or the TFEB protein.

In the instant invention, lysosomal storage disorders are intended as inherited diseases in which a defect in one of many proteins participating in lysosomal biogenesis or metabolism leads to the intralysosomal storage of undegraded molecules, as described in “Lysosomes”, author: Paul Saftig, Landes Bioscience, 2005.

It is an object of the invention a molecule being able to enhance the cellular degradative pathways to prevent or antagonize the accumulation of toxic compounds in a cell, characterized by:

a) acting either directly or indirectly on a CLEAR element to enhance the expression of at least a gene involved in cellular degradative pathways, said CLEAR element comprising at least one repeat of a nucleotide sequence having Seq Id No. 110 as consensus sequence; and
b) belonging to the group of: the TFEB protein, synthetic or biotechnological functional derivative thereof, peptide fragments thereof, chimeric molecules comprising the TFEB protein, synthetic or biotechnological functional derivative thereof; modulator of the TFEB protein activity and/or expression level.

For the TFEB protein it is intended the NCBI GeneID=7942; nt=NM_{—}007162.1, protein=NP_{—}009093.1 (aa. 1-476 of Seq Id No. 228), and variants thereof.

In a particular aspect of the invention the CLEAR element comprises at least one repeat of a nucleotide sequence having Seq Id No. 111 as consensus sequence.

Preferred CLEAR elements are those comprising at least one repeat of a nucleotide sequence selected from the group from Seq Id No. 1 to Seq Id No. 109, most preferred CLEAR elements are those comprising at least one repeat of a nucleotide sequence selected from the group of: Seq Id No. 3, Seq Id No. 9, Seq Id No. 13, Seq Id No. 26, Seq Id No. 28, Seq Id No. 30, Seq Id No. 32, Seq Id No. 34, Seq Id No. 36, Seq Id No. 47, Seq Id No. 50, Seq Id No. 53, Seq Id No. 59, Seq Id No. 62, Seq Id No. 77, Seq Id No. 78, Seq Id No. 84, Seq Id No. 85, Seq Id No. 88, Seq Id No. 92, Seq Id No. 94, Seq Id No. 95, Seq Id No. 98, Seq Id No. 108. Such sequences belong to genes that are responsive either by microarray and/or realtime PCR experiments.

In a particular aspect of the invention the chimeric molecule comprises the TFEB protein and a nuclear localization signal (NLS), more preferably the chimeric molecule has the sequence of Seq Id No. 228.

In another particular aspect of the invention, the modulator of the TFEB protein is a microRNA or a microRNA inhibitor, preferably the modulator of the TFEB protein is the miR-128 or a miR-128 inhibitor.

In a preferred aspect, the molecule of the invention acts either directly or indirectly on a CLEAR element to enhance the expression of at least a gene expressing a lysosomal protein, involved in cellular degradative pathways.

In a preferred aspect, the molecule of the invention is for medical use.

In a preferred aspect, the molecule of the invention is for neurodegenerative disorders' treatments.

Neurodegenerative diseases comprise but are not limited to the following: Alzheimer's disease, Parkinson's disease, Huntington's disease, Creutzfeldt-Jakob disease, Spinocerebellar Ataxia (SCA).

Preferably the neurodegenerative disorder belongs to the group of Alzheimer, Parkinson and Huntington diseases.

In an alternative preferred aspect, the molecule of the invention is for lysosomal storage disorders' treatments.

Lysosomal storage disorders comprise but are not limited to the following: Activator Deficiency/GM2 Gangliosidosis; Alpha-mannosidosis; Aspartylglucosaminuria; Cholesteryl ester storage disease; Chronic Hexosaminidase A Deficiency; Cystinosis; Danon disease; Fabry disease; Farber disease; Fucosidosis; Galactosialidosis; Gaucher Disease (including Type I, Type II, and Type III); GM1 gangliosidosis (including Infantile, Late infantile/Juvenile, Adult/Chronic); I-Cell disease/Mucolipidosis II; Infantile Free Sialic Acid Storage Disease/ISSD; Juvenile Hexosaminidase A Deficiency; Krabbe disease (including Infantile Onset, Late Onset); Metachromatic Leukodystrophy; Pseudo-Hurler polydystrophy/Mucolipidosis IIIA; MPSI Hurler Syndrome; MPSI Scheie Syndrome; MPS I Hurler-Scheie Syndrome; MPS II Hunter syndrome; Sanfilippo syndrome Type A/MPS III A; Sanfilippo syndrome Type B/MPS III B; Sanfilippo syndrome Type C/MPS III C; Sanfilippo syndrome Type D/MPS III D; Morquio Type A/MPS IVA; Morquio Type B/MPS IVB; MPS IX Hyaluronidase Deficiency; MPS VI Maroteaux-Lamy; MPS VII Sly Syndrome; Mucolipidosis I/Sialidosis; Mucolipidosis IIIC; Mucolipidosis type IV; Multiple sulfatase deficiency; Niemann-Pick Disease (including Type A, Type B, and Type C); Neuronal Ceroid Lipofuscinoses, including CLN6 disease; Atypical Late Infantile, Late Onset variant; Early Juvenile Batten-Spielmeyer-Vogt/Juvenile NCL/CLN3 disease; Finnish Variant Late Infantile CLN5; Jansky-Bielschowsky disease/Late infantile CLN2/TPP1 Disease; Kufs/Adult-onset NCL/CLN4 disease; Northern Epilepsy/variant late infantile CLN8; Santavuori-Haltia/Infantile CLN1/PPT disease; Beta-mannosidosis; Pompe disease/Glycogen storage disease type II; Pycnodysostosis; Sandhoff disease/Adult Onset/GM2 Gangliosidosis; Sandhoff disease/GM2 gangliosidosis; Infantile Sandhoff disease/GM2 gangliosidosis; Juvenile Schindler disease; Salla disease/Sialic Acid Storage Disease; Tay-Sachs/GM2 gangliosidosis; Wolman disease.

Preferably the lysosomal storage disorder belongs to the group of Pompe disease and Multiple Sulfatase Deficiency (MSD).

It is another aspect of the invention a nucleic acid containing a sequence encoding for the molecule according as above disclosed,

It is another aspect of the invention a vector comprising under appropriate regulative sequence the above nucleic acid, preferably for gene therapy.

The invention shall be described with reference to experimental non limitating evidences.

### FIGURE LEGENDS

FIG. 1. A regulatory gene network controlling the expression of lysosomal genes. (A) Genomic distribution of CLEAR elements (red spots) at human gene promoters. Scores are assigned based on the CLEAR position weight matrix. Blue spots indicate CLEAR elements in the promoters of lysosomal genes. Dashed box contains all the elements corresponding to the genes that were used for Gene Ontology analysis (see text). (B) Luciferase assay using constructs carrying four tandem copies of either intact (upper) or mutated (middle, mutations in red) CLEAR elements. (C) Expression analysis of lysosomal genes following TFEB overexpression and silencing. Blue bars show the fold change of the mRNA levels of lysosomal genes in TFEB- vs. pcDNA3-transfected cells. Red bars show the fold change of mRNA levels in mimic-miR-128-transfected cells vs. cells transfected with a standard control microRNA (mimic-miR-cel-67). Randomly chosen non-lysosomal genes were used as controls. Gene expression was normalized relative to GAPDH. (D) Chromatin immunoprecipitation (ChIP) analysis. The histogram shows the amount of the immunoprecipitated DNA expressed as percentage of total input DNA. Controls include promoters of housekeeping genes (ACTB, APRT, HPRT), random genes lacking CLEAR sites (TXNDC4, WIF1) and intronic sequences (int) of lysosomal genes. Lysosomal genes and controls were significantly different: Mann-Whitney-Wilcoxon test (P<10-4). All experiments in (B), (C) and (D) were performed in triplicates (data represent mean±s.d.). (E) Confocal microscopy showing colocalization of C1orf85-Myc (green) with the lysosomal membrane marker LAMP1 (red) in HeLa cells.

FIG. 2. TFEB overexpression induces lysosomal biogenesis. Comparison of HeLa stable transfectants of either TFEB or empty pcDNA3 vector (control). (A) Confocal microscopy after staining with an antibody against the lysosomal marker LAMP1. (B) FACS analysis after staining with lysosome-specific dye Lysotracker. The analysis was performed on four independent clones (TFEB#1-4) (see FIG. 18). Blue bars indicate the proportion of cells with fluorescence intensity greater than the indicated threshold (P4 gate). 30,000 cells per clone were analyzed. (C) Electron microscopy analysis. Thin sections exhibit more lysosome profiles (arrows) with typical ultrastructure (see details in inset corresponding to dash boxed area) in TFEB overexpressing transfectants over the control. Scale bar, 720 nm. (D) Number of lysosomes in thin sections (average±s.e., N=20 cells).

FIG. 3. The CLEAR network is activated by lysosomal storage. (A) ChIP analysis following lysosomal storage of sucrose. The histogram shows the ratio (expressed as fold change) between the amounts of FLAG-immunoprecipitated chromatin in sucrose-treated versus non-treated cells. Lysosomal genes show an average two- to three-fold increase of immunoprecipitated chromatin, whereas no significant changes are observed for control genes. (B) Expression analysis of lysosomal genes following sucrose supplementation. The diagram shows a time-course analysis of the mRNA levels of lysosomal genes and of TFEB. Gene expression was monitored by real-time qPCR and normalized relative to GAPDH. All experiments in (A) and (B) were performed at least in duplicates (data represent mean±s.d.). (C) Immunofluorescence microscopy analysis of TFEB subcellular localization following sucrose supplementation. HeLa clones stably expressing TFEB-3×FLAG were stained with an anti-FLAG antibody at various time points after the addition of sucrose in culture medium. (D) Immunofluorescence microscopy analysis of TFEB localization in mouse embryonic fibroblasts (MEFs) from mouse models of three different types of LSDs. MEFs from LSD or wild-type (WT) mice were transiently transfected with a TFEB-3×FLAG construct and stained with an anti-FLAG antibody. The percentages of nuclei positive for FLAG staining were estimated by examining 100 cells per cell type in two different transfection experiments (data represent mean±s.d.).

FIG. 4. TFEB enhances cellular clearance. (A) Comparison of the kinetics of GAG clearance in HeLa stable clones of either TFEB or empty pcDNA3 vector (control). The graph shows relative amounts of 3H-glucosamine incorporated into GAGs over time. 1=3H-glucosamine levels at time zero. Asterisk, P<0.05. Experiments were performed in triplicates (data represent mean±s.d.). (B and C) Clearance of polyQ expanded huntingtin (HTT) following TFEB overexpression. (B) Immuno blot analysis of TFEB-EGFP-positive (+) and TFEB-EGFP-negative (−) HD43 cells separated by FACS 24 h after electroporation. The graph of densitometric analysis shows a strong decrease of polyQ expanded huntingtin in TFEB-EGFP-positive cells compared to controls. (C) Immunocytochemical analysis of TFEB and HTT in HD43(Q105) cells transfected with 3×FLAG-TFEB construct showing little huntingtin staining in cells positive for 3×FLAGTFEB staining.

FIG. 5 Lysosomal genes display coordinated expression behaviour. The diagram reports a visual representation of the expression correlation of 40 lysosomal disease genes with all known lysosomal genes. Each column represents the ˜22,500 gene probes of the Affymetrix HG-U133A platform ranked by their correlation of expression with the gene indicated at the top. Blue bars represent the position of lysosomal genes within the ranked lists. The analysis shows that there is an enrichment of lysosomal genes within the first 5th percentile of ranked lists of expression correlation.

FIG. 6 Detailed view of the expression correlation among lysosomal genes. The columns include the first 100 gene probes of the expression correlation lists for selected lysosomal genes. Lysosomal genes are highlighted in orange. Other genes associated to the lysosomal function are highlighted in yellow. It should be noted that in a randomly ranked list the probability of finding a lysosomal gene probe is ˜1:100.

FIG. 7 Logo representation of the CLEAR element. The conservation of each residue within columns is visualized as the relative height of symbols.

FIG. 8 Distribution of CLEAR elements at the promoter regions of a subset of lysosomal genes. The CLEAR elements are clustered, often in multiple copies, around the transcription start site. The legend to colour code is reported as a schematic diagram in the figure.

FIG. 9 Enzymatic activities. Quantification of the activities of lysosomal enzymes β-glucosidase, cathepsin D and β-glucuronidase in HeLa cells stably overexpressing TFEB and controls. Asterisk, P<0.05. All measures were performed in triplicates (data represent mean±s.d.).

FIG. 10 Expression analysis of lysosomal genes following TFEB overexpression in HEK293 cells. Blue bars show the fold change of the mRNA levels of monitored genes in TFEB- vs. pcDNA3-transfected cells. Gene expression was normalized relative to GAPDH.

FIG. 11 Validation of TFEB as a target gene of miR-128 by dual luciferase assay. The 3′UTR region of TFEB was cloned into a firefly luciferase sensor construct and transfected into HeLa cells along with a Renilla Luciferase control. Luciferase activities were measured in the presence or absence of a plasmid construct containing the precursor sequence of hsa-miR-128. EZH2 and LRIG1 genes, which were not predicted targets of miR-128, were used as negative controls. All experiments were performed in triplicates (data represent mean±s.d.).

FIG. 12 Expression analysis of lysosomal genes following mimic-miR-128 transfection into HeLa cells stably expressing a TFEB transgene lacking the 3′UTR region. To verify that the downregulation of lysosomal genes following mimic-miR-128 transfection was due to TFEB silencing, mimic-miR-128 was transfected into HeLa clones stably expressing a TFEB transgene lacking the TFEB 3′UTR region, which contains the miR128 binding site. Blue bars show the fold change of monitored genes in mimic-miR-128-transfected cells vs. cells transfected with a standard control microRNA (mimic-miR-cel-67). No significant changes were observed for any of the genes tested. Gene expression was normalized relative to GAPDH.

FIG. 13 Analysis of transcriptome changes following TFEB transient transfection in HeLa cells. The graph shows a Gene Ontology analysis by ‘Cellular Compartment’ category of up regulated genes with false discovery rate<0.1.

FIG. 14 Venn diagram showing the overlap between lysosomal genes and genes induced by TFEB overexpression in HeLa cells at an FDR<0.10. The diagram shows that 20 genes, all containing CLEAR sites in their promoters, are represented in both categories. This is likely to be an underestimate as it is based on highly stringent statistical criteria and on a single cell type. A more comprehensive view of the response of lysosomal genes to TFEB induction is shown in FIG. 15 (Gene Set Enrichment Analysis).

FIG. 15 Gene Set Enrichment Score Analysis (GSEA) of transcriptome changes following TFEB overexpression. The graph shows the enrichment plots generated by GSEA analysis of ranked gene expression data (left: upregulated, red; right: down-regulated, blue). The enrichment score is shown as a blue line, and the vertical blue bars below the plot indicate the position of lysosomal genes carrying CLEAR sites in their promoters. The analysis shows that lysosomal genes with CLEAR sites are mostly grouped in the fraction of up-regulated genes (Enrichment Score=0.84; P<0.0001).

FIG. 16 FACS analysis after staining with lysosome-specific dye lysotracker of HeLa stable transfectants of TFEB (TFEB#1-4). Blue bars indicate the proportion of cells with fluorescence intensity greater than the indicated threshold (P4 gate). 30,000 cells per clone were analyzed.

FIG. 17 Microscopy analysis of MSD cells at 48 hours following the transfection of an empty vector (left) or a TFEB vector (right). The arrows indicate the storage of glycosaminoglycans in untreated MSD cells. The experiment shows that cells treated with TFEB no longer display accumulation of undigested glycosaminoglycans.

FIG. 18 Electron microscopy analysis of MSD cells at 48 hours following the transfection of an empty vector (left) or a TFEB vector (right). Untreated cells show an extensive vacuolization due to the storage of undigested glycosaminoglycans. Cells treated with TFEB show that the cellular vacuolization is largely reversed.

FIG. 19 Immunofluorescence analysis of Pompe disease cells treated with a TFEB-3×FLAG vector. Transfected cells (arrows) show a strong reversal of the extensive vacuolization found in non-transfected cells (on the right) due to the accumulation of glycogen.

FIG. 20 Inhibition of miR-128 results in the transcriptional activation of the CLEAR network. Cultured HeLa cells were transfected with a specific inhibitor of miR-128 (Dharmacon) or with a standard control (inhibitor of miR-cel-167) that has no target in human cells. Real-time qPCR was performed to monitor the expression of TFEB, its lysosomal target PSAP, two housekeeping genes (HPRT and GAPDH) and two random genes (ARPP-19 and HOXA9) 48 hours after transfection. The graph shows the ratio between the expression levels of monitored genes in cells transfected with the inhibitor of miR-128 versus control. The results show an increase in the expression of both TFEB and its target PSAP, and no changes in control genes. Gene expression was normalized relative to HPRT.

FIG. 21 Amino acid sequence of the engineered analog of TFEB, TFEB-NLS (Seq Id No. 228). TFEB-NLS was obtained by the addition of a nuclear localization signal (NLS) at the C-terminus of the protein. The nuclear localization signal has sequence PKKKRK (underlined in the figure).

FIG. 22 TFEB-NLS localizes in the nucleus. Immunofluorescence analysis of the TFEB analog TFEB-NLS showing a complete nuclear localization of the TFEB-NLS construct. Two series of images are reported as representative of the subcellular localization of TFEB-NLS. In each series, on the left cell nuclei are stained with the DAPI dye (specific for the DNA); on the right, cells are stained for TFEB.

### MATERIAL AND METHODS

### Genome Analysis

Human genomic sequences were retrieved from the Ensemble database (http://www.ensembl.org) and analyzed by using the Regulatory Sequence Analysis Tool (28). Iterative analyses led to the identification of a consensus sequence of the CLEAR element. A position weight matrix (PWM) was built by assembling all CLEAR elements found within 200 bp from the transcription start site of lysosomal genes. Human gene promoters were searched with the CLEAR PWM using the PatSer tool (28) with default parameters. Gene Ontology (GO) analyses were performed with the web tool DAVID (http://david.abcc.ncifcrf.gov) using default parameters. Only non-redundant terms with a value≦0.01 and Fold Enrichment≧2 were retained.

### Expression Correlation Analysis

Expression correlation analysis was performed as previously described (29), with minor modifications. Briefly, lysosomal genes were analyzed by using the g:Sorter tool, which is part of the g:Profiler package (30). For a selected gene probe, g:Sorter can retrieve a number of most similar coexpressed profiles in a specified GEO data set. The analysis was carried out on a total of 160 heterogeneous microarray experiments, based on the HG-U133A GeneChip array. g:Sorter was queried with the gene probes for a representative set of lysosomal genes. For each analyzed probe, the first 3% of most correlated gene probes was retrieved for each microarray data set. Subsequently, all HG-U133A gene probes were ranked based on their occurrence in the 160 different lists of most correlated genes. Genes with an equal number of occurrences were sub-ranked according to their average ranking within the various experiments. The procedure resulted in lists of gene probes ranked by their expression correlation to the investigated genes.

### Cell Culture and Transfection

HeLa cells and mouse embryonic fibroblasts from mouse models of MPSII (31), MPSIIIA (32), and MSD (33), were grown in Dulbecco's Modified Eagle's Medium (DMEM, Euroclone), supplemented with 10% heat-inactivated Fetal Bovine Serum (FBS, Hyclone). Where indicated, the medium was supplied with sucrose to a final concentration of 100 mM. Cells were seeded in six-well plates at 10% confluence before transfection. Transfection was performed by using PolyFect Transfection Reagent (Qiagen) or Interferin (PolyPlus transfection) according to the manufacturer's protocols. Transfectants for full-length TFEB and TFEB-3×FLAG were selected with 1 mg/ml G418 (Sigma). For microRNA experiments, cells were transfected with 200 nM miRIDIAN Dharmacon miRNA Mimics (miR-128, or negative control cel-miR-67) and harvested after 48 h for total RNA extraction.

### Luciferase Assays

To test the ability of the CLEAR site to promote transcription, HeLa cells were transfected with pGL3-basic luciferase reporter plasmids containing four tandem copies of either the sequence (4×CLEAR consensus sequences as in Seq Id No. 111 in bold characters) Seq Id No. 112:

CCGGGTCACGTGACCCCAGGGTCACGTGACCCTGCGGGTCACGTGACCCT
GCGGGTCACGTGACCCCC

or the sequence (4×control sequences in bold characters) Seq Id No. 113:

CCGGGAATCGTGACCCCAGGGAATCGTGACCCTGCGGGAATCGTGACCCT
GCGGGAATCGTGACCCCC.

To validate TFEB as a target of miR-128, HeLa cells were transfected with firefly luciferase reporter plasmids containing the 3′UTR regions of either TFEB or control genes (EZH2 and LRIG1) and with a psiUx plasmid (34), construct containing the precursor sequence of hsa-miR-128. Luciferase assays were performed 48 h after transfection using Dual Luciferase Reporter Assay System (Promega), normalized for transfection efficiency by cotransfected Renilla luciferase.

### Molecular Biology

Full-length human MITF, TFE3, TFEB and TFEC were cloned into the pcDNA3.1 vector (Invitrogen). Full-length TFEB was also cloned into the p3×FLAG-CMV-10 vector. Full-length C1orf85 was cloned into the pcDNA3.1/c-Myc vector (Invitrogen). RNA samples were obtained using either the RNeasy or the miRNeasy kit (Qiagen) according to the manufacturer's instructions. RNA was quantified using the NanoDrop 8000 (Thermo Fischer). cDNA was synthesized using QuantiTect Reverse Transcription kit (Qiagen).

### Chromatin Immunoprecipitation Assay (ChIP)

ChIP assays were carried out using formaldehyde-fixed nuclei isolated from HeLa transfectants carrying a TFEB-3×FLAG transgene or a control HeLa cell line without any tagged transgene (mock). Each ChIP experiment required 10⁷ cells. ChIP was performed using the ANTI-FLAG M2 Affinity Gel (Sigma) according to the manufacturer's protocol.

### Quantitative Real-Time PCR

Real-time quantitative RT-PCR on cDNAs or sonicated chromatin was carried out with the LightCycler 480 SYBR Green I mix (Roche) using the Light Cycler 480 II detection system (Roche) with the following conditions: 95° C., 5 min; (95° C., 10 s; 60° C., 10 s; 72° C., 15 s)×40. For expression studies the qRT-PCR results were normalized against an internal control (GAPDH). Oligonucleotide sequences are reported in Table 5.

### Microarray Experiments

Total RNA from TFEB-transfected HeLa cells was used to prepare cDNA for hybridization to the Affymetrix Human Gene 1.0 ST array platform. Hybridizations were performed in triplicates at the Coriell Genotyping and Microarray Center, Coriell Institute for Medical Research, Camden, N.J., USA. A false discovery rate<0.1 was used to assess significant gene differential expressions. Gene Set Enrichment Analysis was performed as previously described (35). The cumulative distribution function was constructed by performing 1,000 random gene set member-ship assignments. A nominal P value<0.01 and an FDR<10% were used to assess the significance of the Enrichment Score (ES).

### Confocal Imaging

Transfected HeLa cells were grown on glass coverslips for 24 h, washed with PBS containing 100 mM MgCl₂ and 100 mM CaCl₂ (PBS/Ca/Mg), and fixed with 4% paraformaldehyde (PFA; Sigma) for 10 min. After washing and quenching PFA with 50 mM NH₄Cl for 15 min, cells were washed with PBS and permeabilized in blocking buffer (0.05% saponin/0.2% BSA in PBS/Ca/Mg) for 20 min. Coverslips were then incubated O/N with appropriate primary antibodies and for 1 h with Alexa-594 and Alexa-488 conjugated secondary antibodies (Molecular Probes). Coverslips were mounted on glass slides with Vectashield (Vector Laboratories). Images were taken using a confocal microscope (LSM510; Carl Zeiss, Inc.) using a Plan-Neofluar 63× immersion objective (Carl Zeiss, Inc.).

### Electron Microscopy

Control and TFEB-overexpressing HeLa cells were washed with PBS, and fixed in 1% glutaraldehyde dissolved in 0.2 M Hepes buffer (pH 7.4) for 30 min at room temperature. The cells were then postfixed for 2 h in OsO₄. After dehydration in graded series of ethanol, the cells were embedded in Epon 812 (Fluka) and polymerized at 60° C. for 72 h. Thin sections were cut at the Leica EM UC6, counterstained with uranyl acetate and lead citrate. EM images were acquired from thin sections using a Philips Tecnai-12 electron microscope equipped with an ULTRA VIEW CCD digital camera (Philips, Eindhoven, The Netherlands). Quantification of lysosomes was performed using the AnalySIS software (Soft Imaging Systems GmbH, Munster, Germany). Selection of cells for quantification was based on their suitability for stereologic analysis, i.e. only cells sectioned through their central region (detected on the basis of the presence of Golgi membranes) were analyzed. Lysosomal profiles were detected on the basis of typical ultrastructural characteristics such as high electron density, presence of multiple internal luminal vesicles, concentric and myelinoid bodies.

### Huntingtin Clearance

Huntingtin inducible striatal cells [HD43(Q105)] were cultured at 33° C. in DMEM high glucose, supplemented as described previously (36). HD43(Q105) cells were electroporated with a pCIG2-TFEB vector containing an IRES2-EGFP cassette, or with an empty pCIG2 vector as a control, using a Gene Pulser II electoporator (BioRad). Immediately after the electoporation, cells were plated in presence of 0.2 μg/ml doxycycline (Sigma) in order to induce the transgene for expanded huntingtin. Twenty-four hours post-induction, GFP-positive cells were sorted by flow cytometry using the BD FACSAria cytometer (BD Biosciences) and used for immuno blot analysis.

### FACS Analysis

Cells were kept in 50 nM acidotropic dye LysoTracker Red DND-99 (Molecular Probes) for 40 min. Red lysosomal fluorescence of 30,000 cells per sample was determined by flow cytometry using the BD FACSAria cytometer (BD Biosciences).

### GAG Clearance

HeLa cells were grown in RPMI medium (Gibco, Invitrogen, Grand Island) supplemented with 10% FCS in the presence of 7 μCi/ml ³H-glucosamine hydrochloride (Perkin Elmer, 37.75 Ci/mmol, Boston) for 3 days, washed extensively with PBS and chased for variable times. At each time point cells were harvested, homogenized and subject to chromatography on Sephadex G-25 columns (GE Healthcare, Sweden) to eliminate unincorporated ³H-glucosamine hydrochloride. The amounts of incorporated radioactivity was measured by liquid scintillation in a Beckman L56500 counter (Beckman Instruments, Fullerton, Calif., USA).

### Immuno-Blot

Cells were lysed in cold lysis buffer (20 mM Tris-HCl, pH 7.4, 150 mM NaCl, 1% TritonX-100) in the presence of protease inhibitors (SIGMA) for 30 min on ice. 20 mg of protein samples were separated on SDS-PAGE acrylamide gel and transferred onto nitrocellulose membrane (Amersham Pharmacia Biotech). Primary and (HRP)-conjugated antibodies were diluted in 1% BSA TBS-T. Bands were visualized using the ECL detection reagent (Pierce) and normalized against actin. Proteins were quantified by the Bradford method. Antibodies: Huntingtin, MAb2166 (Chemicon, Temecula, Calif.); Actin (Sigma).

### Enzymatic Activities

Cathepsin D activity was determined with the Cathepsin D Assay Kit (Sigma) following manufacturer's instructions. β-glucosidase activity was determined by incubating cell homogenates (10⁷ cells, ˜10 μg proteins) with 5 mM 4-MU-beta-D-glucopyranoside in 0.1 M acetate buffer, pH 4.2, for 3 hrs at 37° C. β-glucuronidase activity was determined by incubating cell homogenates (2.5×10⁷ cells, ˜25 μg proteins) with 10 mM 4-MU-glucuronide in 0.2 acetate buffer, pH 4.8, for 1 hr at 37° C. Both reactions were stopped with 1 ml glycine-carbonate buffer, pH 10.7. Fluorescence was read at 365 nm (excitation) and 450 nm (emission) on a Turner Modulus fluorometer.

### Data Analysis

Most data are presented as the mean±s.d. Statistical comparisons were made using analysis of variance (ANOVA). A P value<0.05 was considered statistically significant.

### Results

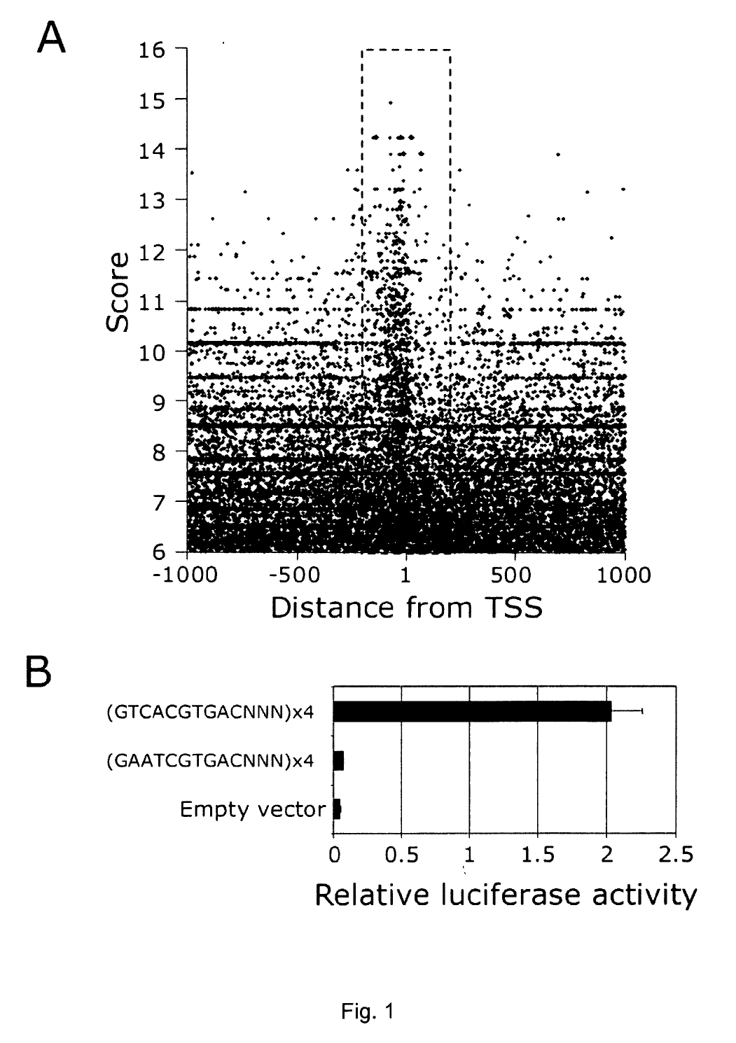

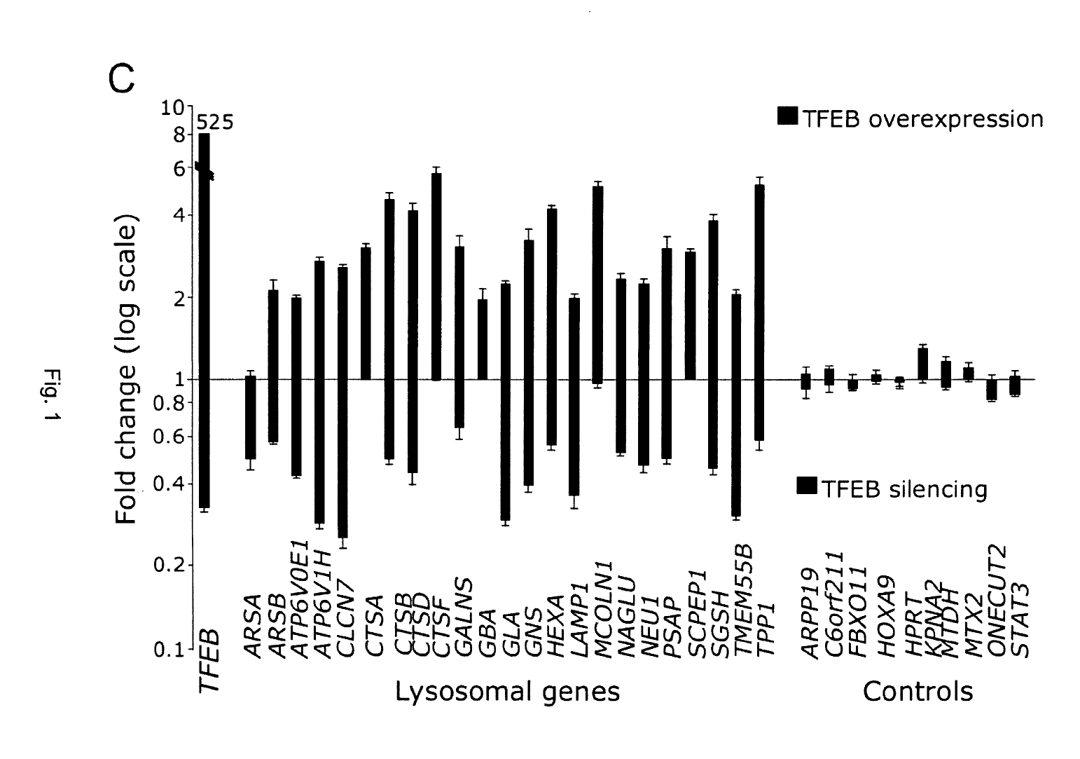

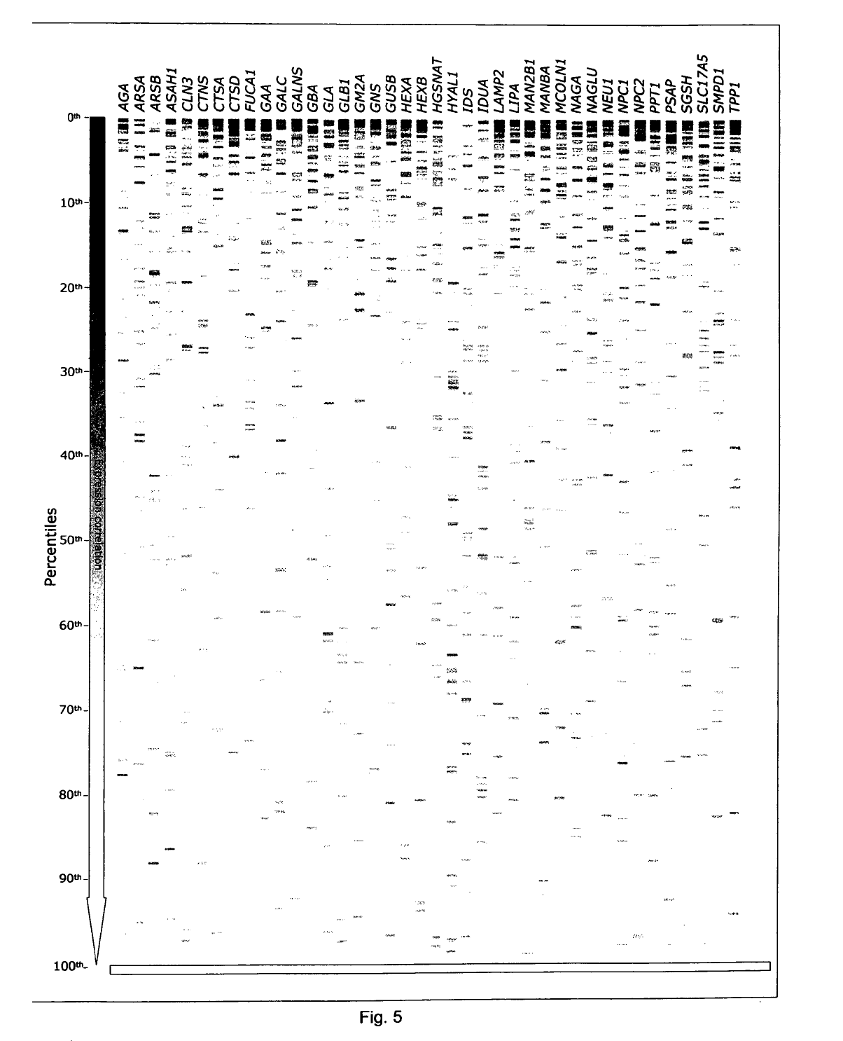

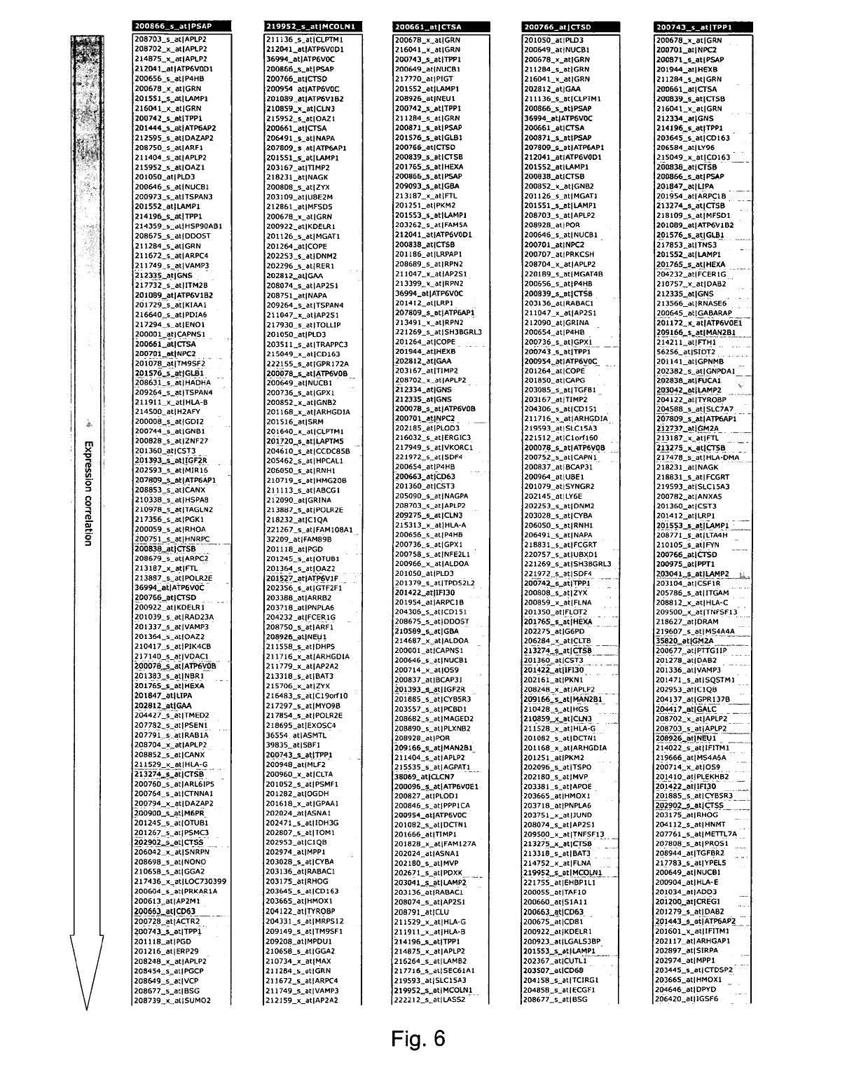

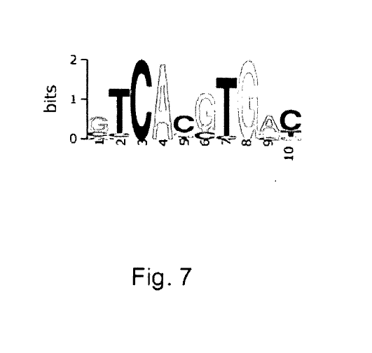

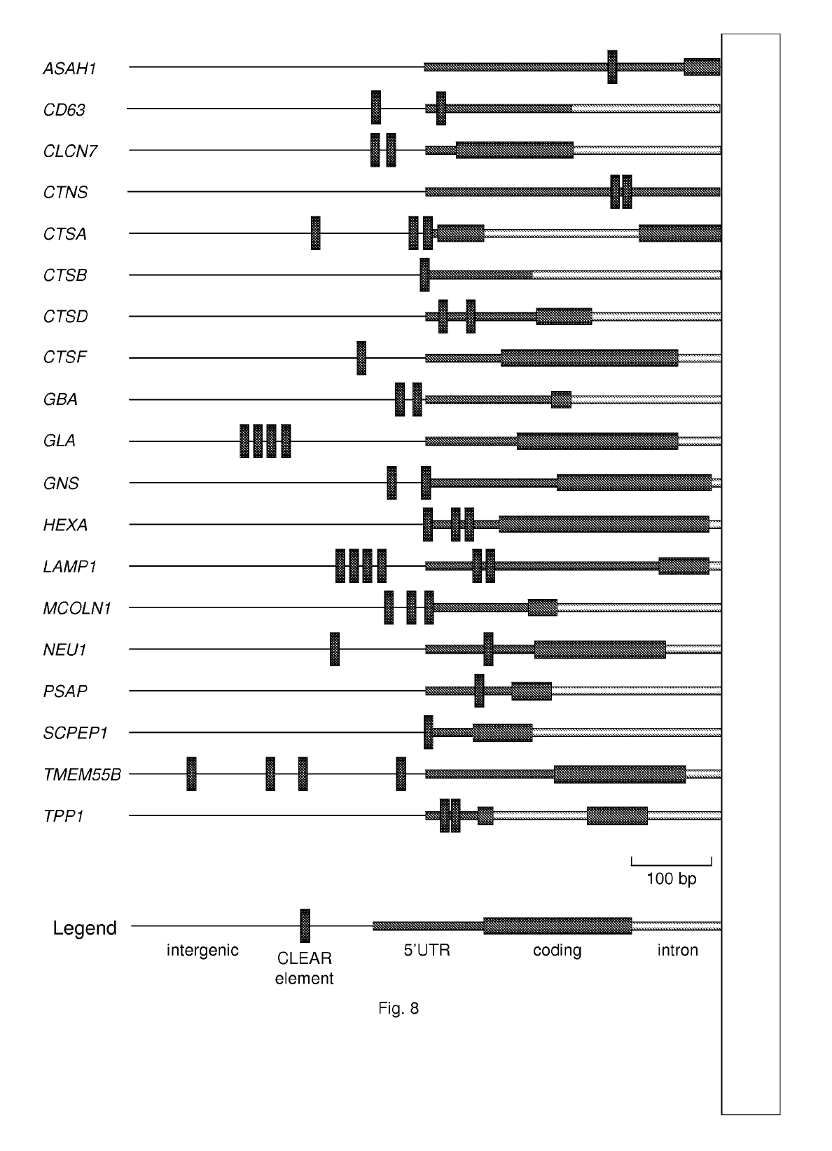

As stated above, lysosomes are specialized to degrade macromolecules received from the secretory, endocytic, autophagic and phagocytic pathways (1). As degradation requirements of the cell may vary depending on tissue type, age, and environmental conditions, authors postulated the presence of a cellular program coordinating lysosomal activity. By using the g:profiler (2) tool authors observed that genes encoding lysosomal proteins, hereafter referred to as lysosomal genes, tend to have coordinated expression (FIGS. 5 and 6). Pattern discovery analysis of the promoter regions of the 96 known lysosomal genes (3) resulted in the identification of a palindromic 10-bp GTCACGTGAC motif highly enriched in this promoter set (68 genes out of 96; P<0.0001) (FIG. 7). This motif is preferentially located within 200 bp from the transcription start site (TSS), either as a single sequence or as tandem multiple copies (FIG. 8 and Table 1). The distribution of this motif was determined around all human gene TSSs (FIG. 1A) and gene ontology analysis of the genes with at least two motifs within 200 bp from the TSS—suggesting they are likely in a promoter—showed a significant enrichment for functional categories related to lysosomal biogenesis and function (Table 2). Thus, authors named this motif Coordinated Lysosomal Expression And Regulation (CLEAR) element. A luciferase assay showed that the CLEAR element mediates transcriptional activation (FIG. 1B).

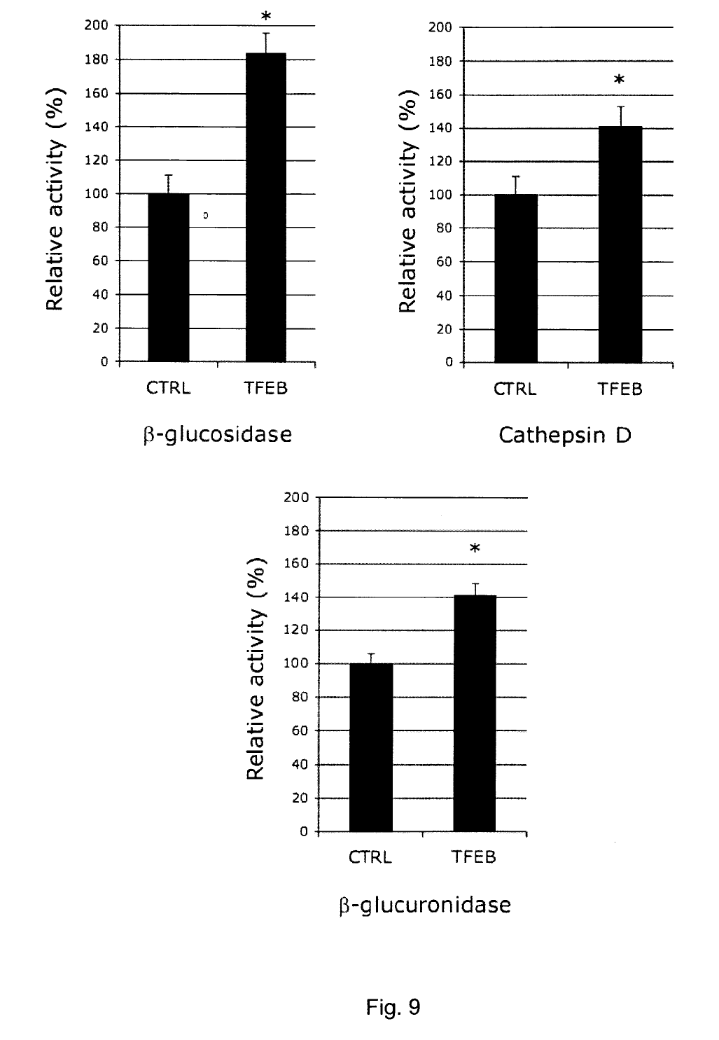

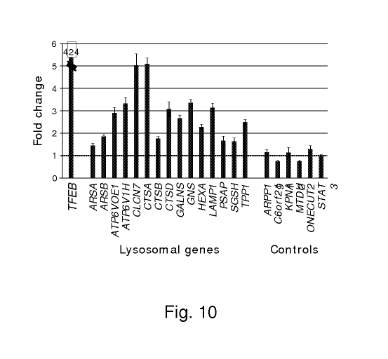

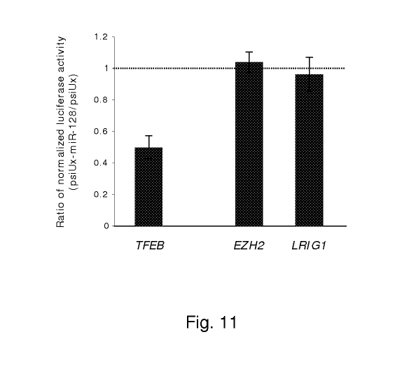

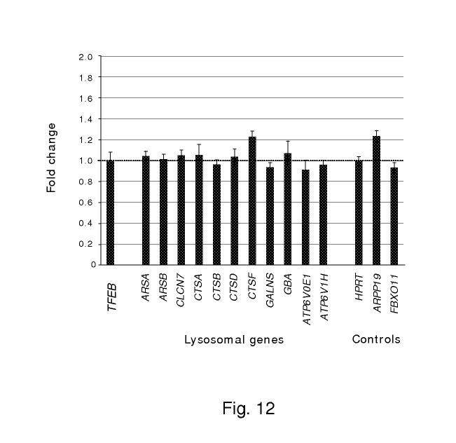

The CLEAR consensus sequence shown as Seq Id No. 110 overlaps that of the E-box (CANNTG), a known target site for bHLH transcription factors (4). In particular, members of the MiT/TFE subfamily of bHLH factors were found to bind sequences similar to the CLEAR consensus (5). The MiT/TFE subfamily is composed of four members in humans: MITF, TFE3, TFEB, and TFEC (6). To determine whether any of these proteins are able to modulate the expression of lysosomal genes, authors transfected HeLa cells with plasmids carrying MITF, TFE3, TFEB, or TFEC cDNAs. Authors observed an increase in the mRNA levels of lysosomal genes (22 out of 23 genes tested) only following TFEB overexpression (FIG. 1C). Accordingly, authors detected a significant increase in the activities of lysosomal enzymes β-glucosidase, Cathepsin D and β-glucuronidase (FIG. 9). Induction of lysosomal genes following TFEB overexpression was also observed in HEK293 cells (FIG. 10). Authors predicted that TFEB could be a target of the micro-RNA miR-128 (7), which was confirmed by luciferase experiments (FIG. 11). MicroRNA-mediated TFEB silencing was associated with the downregulation of 18 out of the 23 lysosomal genes tested (FIGS. 10 and 12). Thus, TFEB regulates the expression of lysosomal genes.

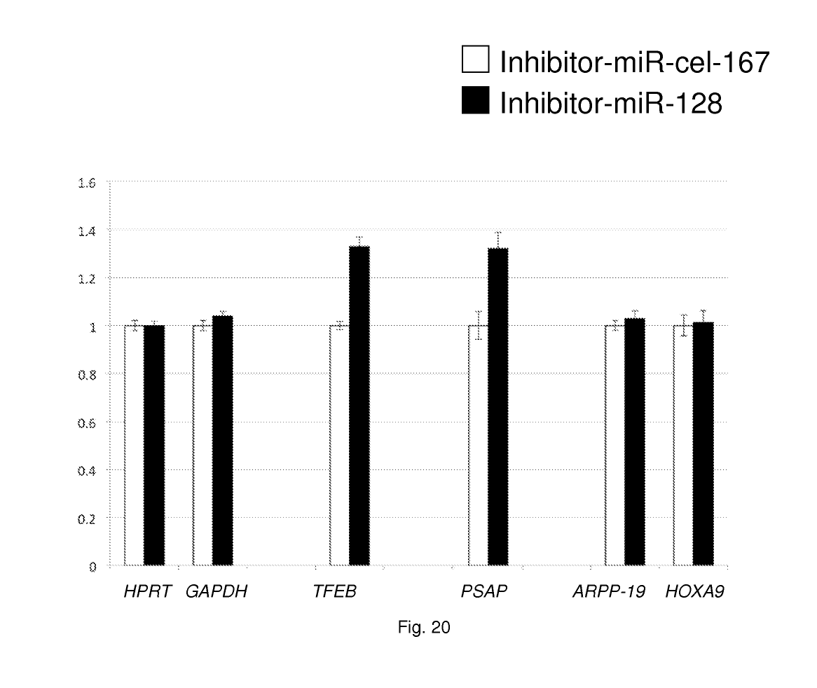

The inhibition of miR-128, performed with a specific miRNA inhibitor (Dharmacon), resulted in the increase of the expression of TFEB and of its target lysosomal gene PSAP (FIG. 20), demonstrating that the modulation of the expression of miR-128 can directly influence the activation of the CLEAR network.

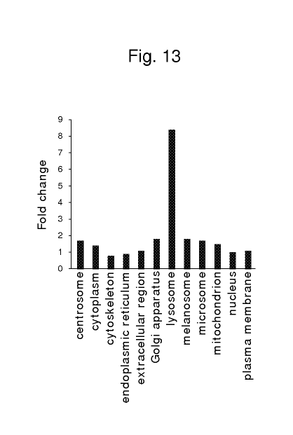

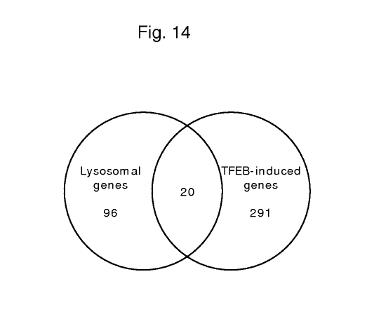

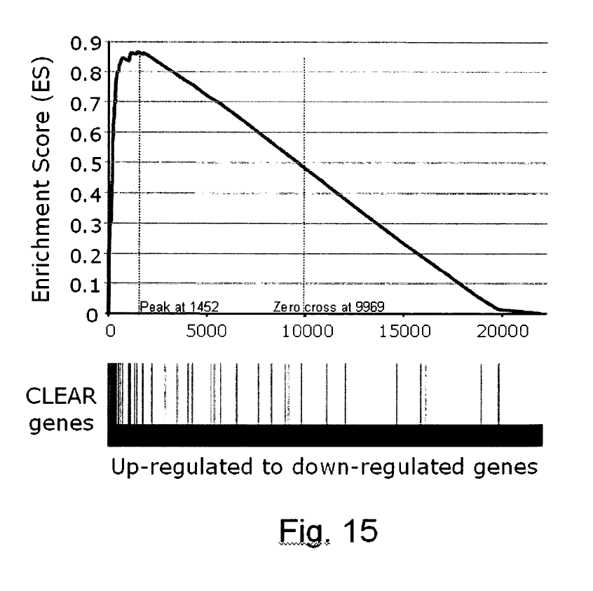

To test whether lysosomal genes are direct targets of TFEB authors performed chromatin immunoprecipitation (ChIP) analysis on HeLa cells stably expressing a TFEB 3×FLAG construct using an anti-FLAG antibody. The results demonstrated that TFEB binds to CLEAR sites (FIG. 1D). To identify genes responsive to TFEB on a genomic scale authors performed microarray analysis of the HeLa transcriptome following TFEB overexpression. Authors observed that 291 genes were up-regulated, and 7 down-regulated, at a false discovery rate<0.1 (Table 3). Up-regulated genes were greatly enriched with lysosomal genes and genes related to lysosomal biogenesis and function (FIGS. 13 and 14, Table 4). Accordingly, Gene Set Enrichment Analysis (GSEA) showed a significant enrichment (Enrichment Score=0.84; P<0.0001) of lysosomal genes that contain CLEAR elements in their promoters among induced genes (FIG. 15). Interestingly, non-lysosomal genes involved in degradation pathways appear to be modulated by TFEB. These include: RRAGC and UVRAG, which are key factors regulating autophagy (8, 9); CSTB, which plays a role in protecting against the proteases leaking from lysosomes (10); M6PR and IGF2R, which mediate the import of proteins into the lysosome (11). To illustrate the feasibility of using the CLEAR network as a tool to identify genes involved in lysosomal function and to provide candidate genes for orphan lysosomal diseases (3), authors determined the subcellular distribution of two randomly chosen proteins of unknown function, C1orf85 and C12orf49. The uncharacterized TFEB target, C1orf85, was found localized to lysosomes (FIG. 1E).

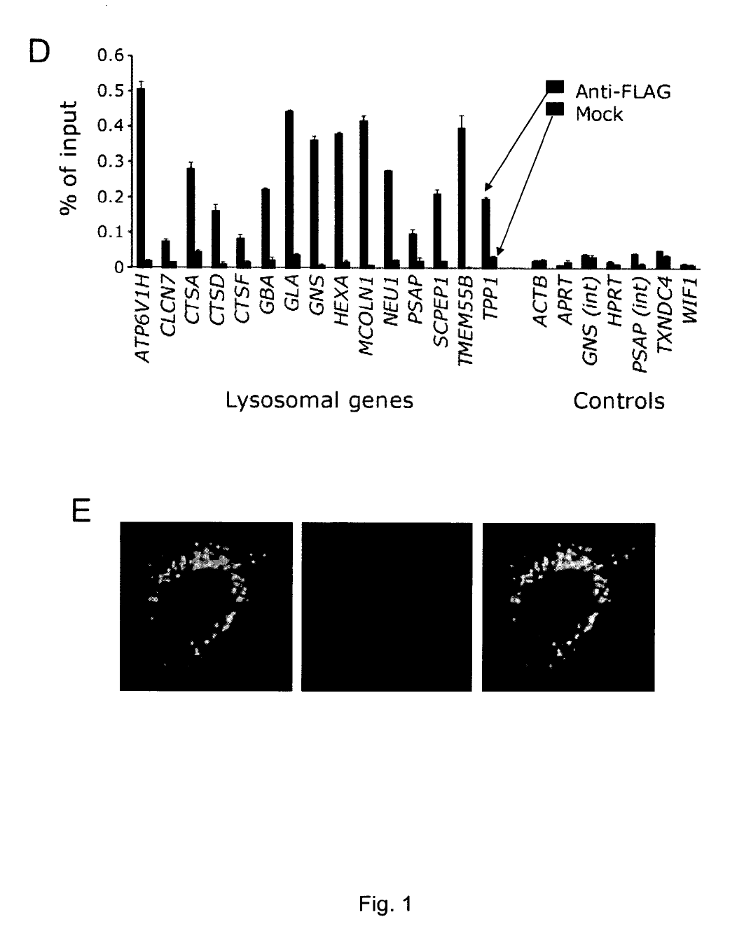

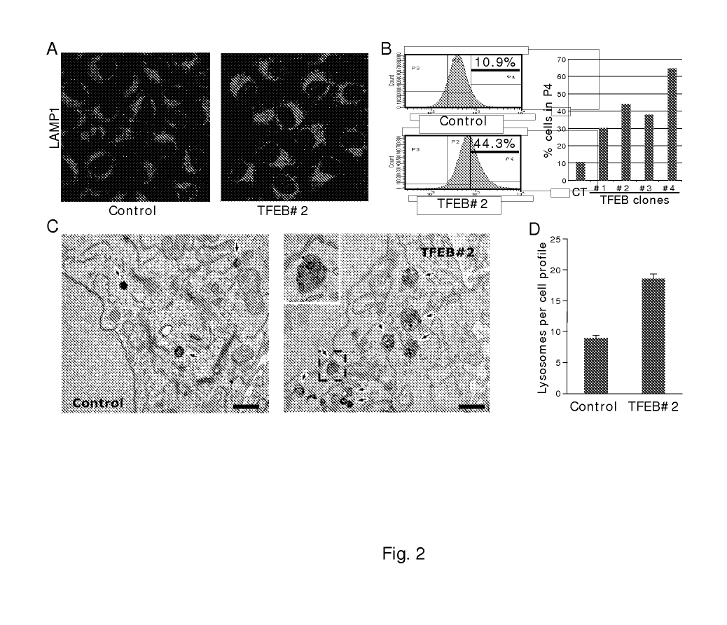

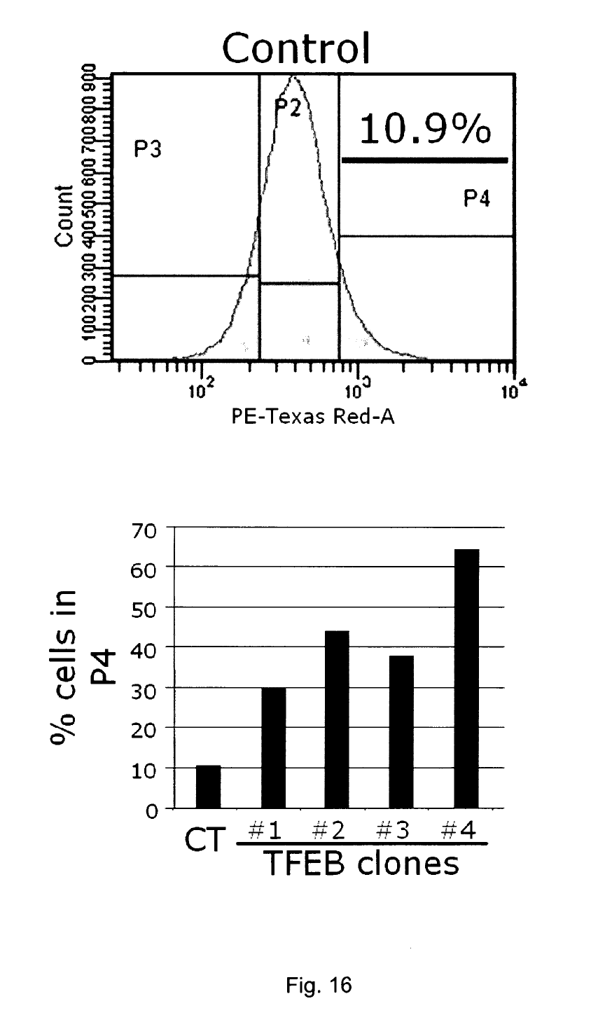

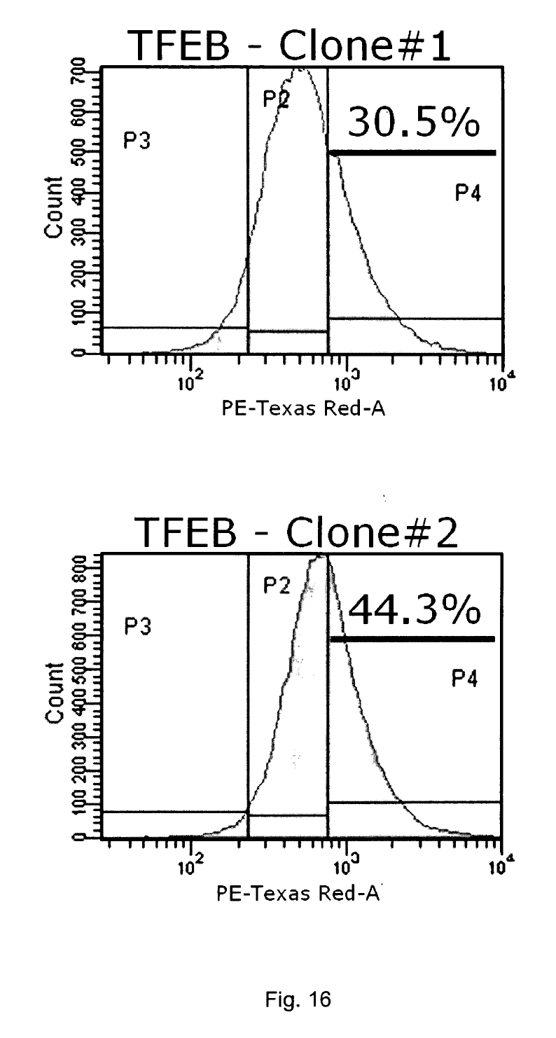

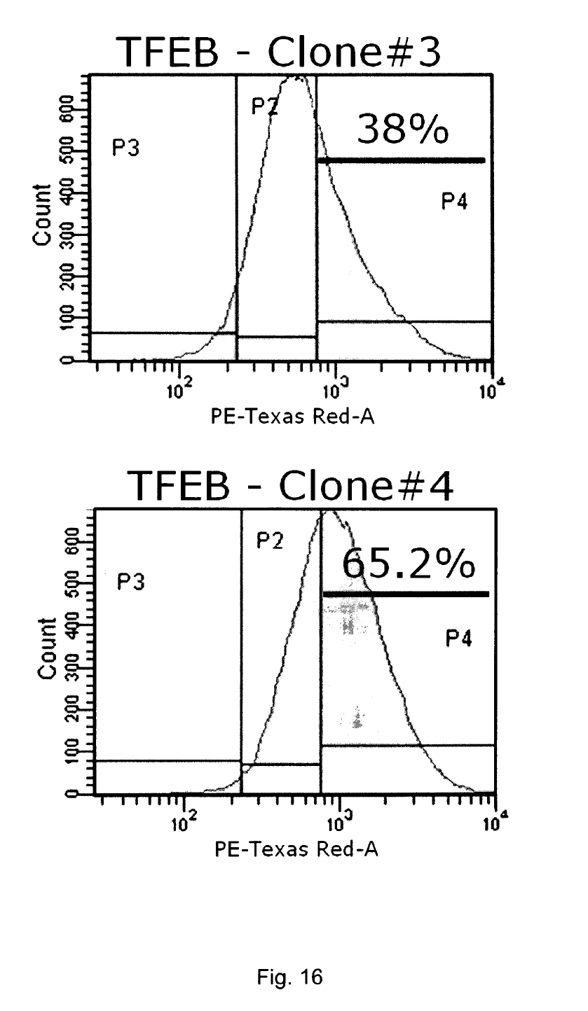

An expansion of the lysosomal compartment was detected in HeLa transfectants stably overexpressing TFEB (FIGS. 2, A and B and FIG. 16). Accordingly, ultrastructural analysis revealed a significant increase in the number of lysosomes per cell (FIGS. 2, C and D), indicating the involvement of TFEB in lysosomal biogenesis.

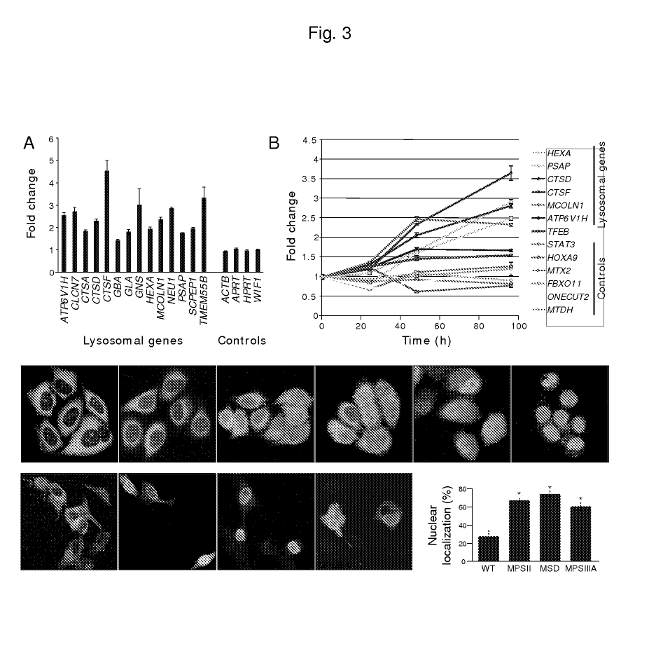

Authors used a sucrose-induced vacuolization model (12, 13) to test whether the TFEB-CLEAR network responds to lysosomal storage of undegraded molecules. An increase of the binding events of TFEB to lysosomal promoters (FIG. 3A) and of the mRNA levels of lysosomal genes, and to a lesser extent of TFEB, was detected upon sucrose supplementation to the culture medium (FIG. 3B). The addition of sucrose also determined the progressive translocation of TFEB from a diffuse localization in the cytoplasm, where it predominantly resides in untreated cells, to the nucleus (FIG. 3C), suggesting that nuclear translocation is an important mechanism for TFEB activation.

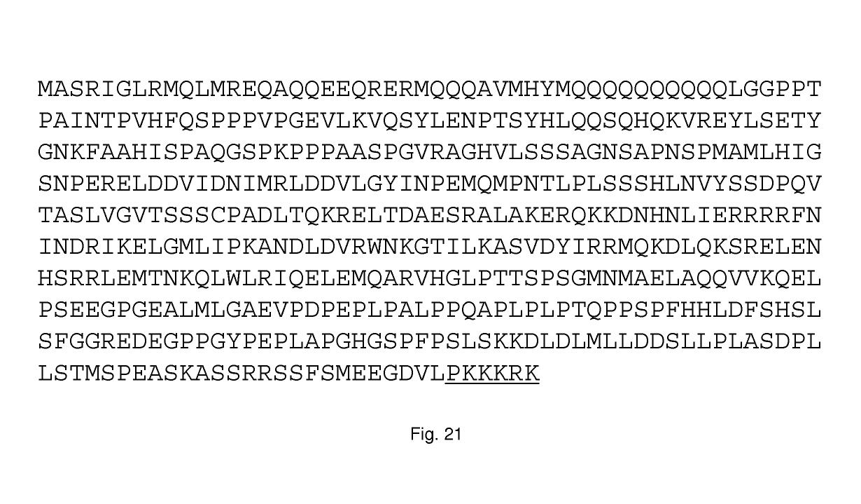

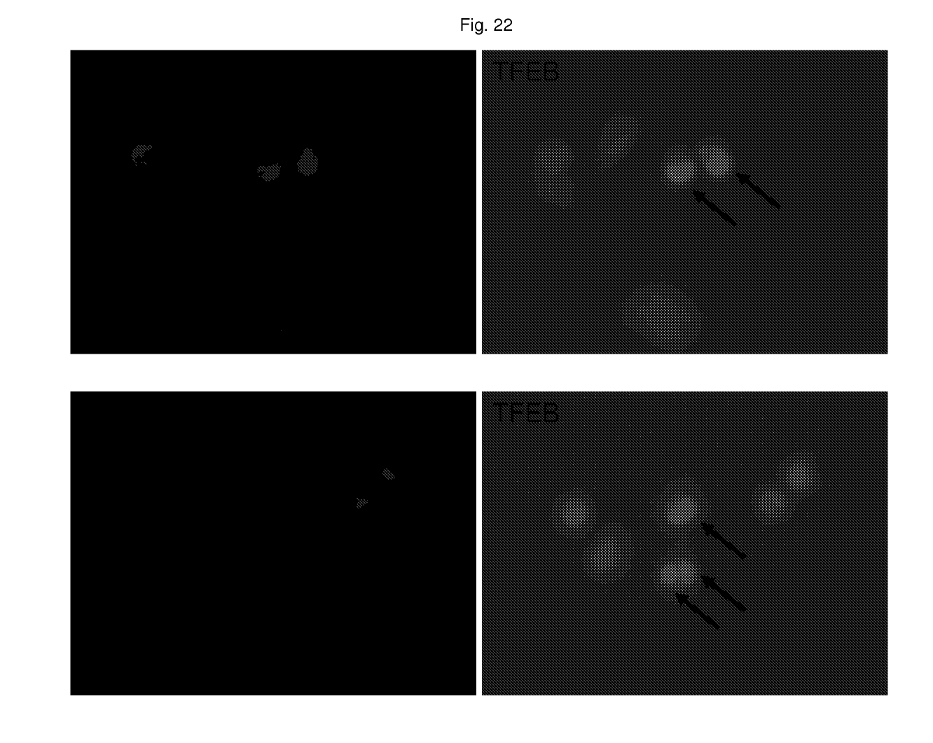

Over 40 lysosomal storage disorders (LSDs) are characterized by the progressive accumulation of undigested macromolecules within the cell, resulting in cellular dysfunction that leads to diverse clinical manifestations (1, 14, 15). Authors investigated TFEB subcellular localization in embryonic fibroblasts obtained from mouse models of three different LSDs, Mucopolysaccharidoses types II and IIIA (MPSII and MPSIIIA) and Multiple Sulfatase Deficiency (MSD) (16-18). A predominant nuclear localization of TFEB was detected in cells from all three LSD mouse models (FIG. 3D), suggesting that the TFEB signaling pathway is activated following the intra-lysosomal storage of undegraded molecules. Such activation could be part of the cellular physiological response to lysosomal stress and could serve degradation needs by enhancing the lysosomal system. In order to obtain a TFEB molecule able to completely and directly localize into the nucleus, authors designed a TFEB analog (chimeric molecule) by adding a nuclear localization signal (NLS) at the C-terminus of the TFEB protein (Seq Id No. 228, FIG. 21). Immunofuorescence analysis of HeLa cells transfected with the TFEB-NLS construct demonstrated that it indeed localize into the nucleus (FIG. 22), with no needs for storage conditions.

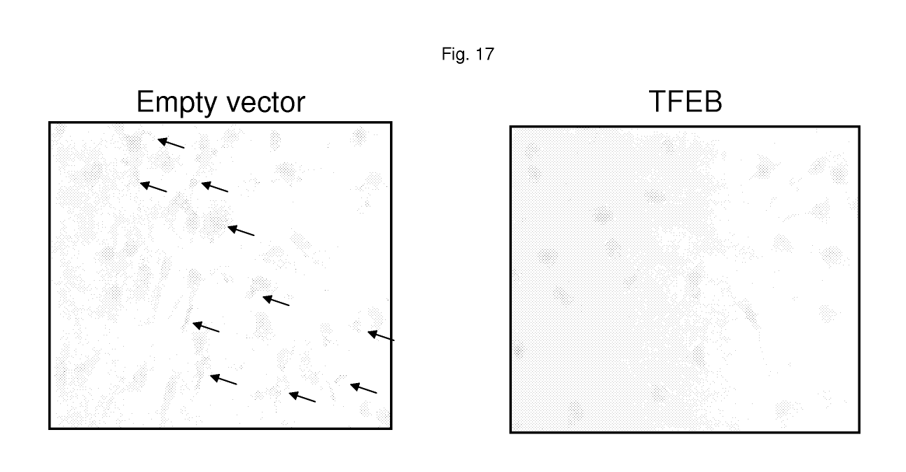

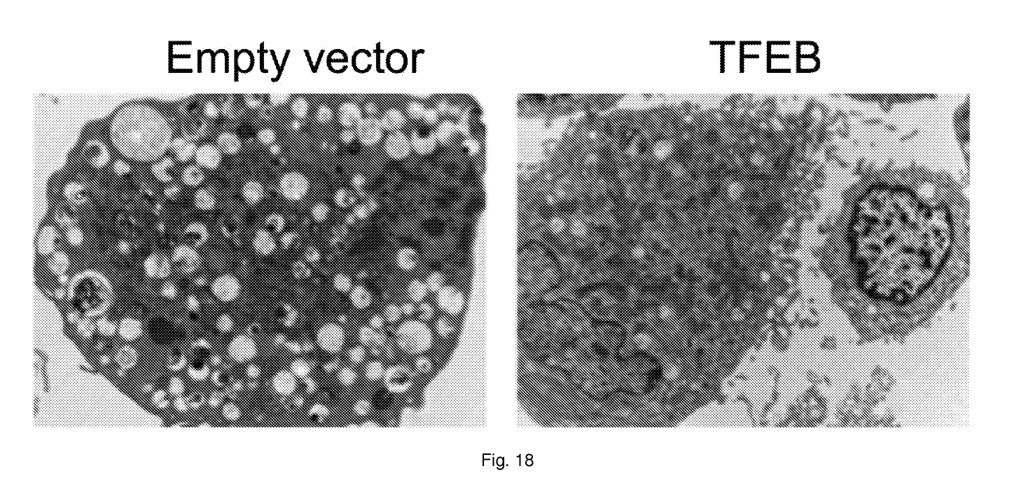

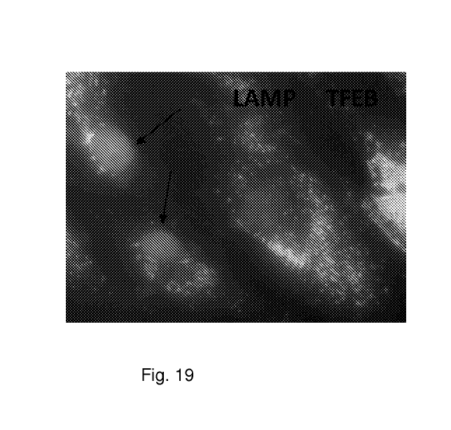

Lysosomal storage disorders are caused by the intracellular accumulation of undigested material due to mutations in genes participating to lysosomal function. In Multiple Sulfatase Deficiency (MSD), a severe human disorder, a defect in sulfatases impairs the ability of the cell to degrade sulfated compounds, with the subsequent accumulation of glycosaminoglycans that induce extensive cellular vacuolization and finally prove to be toxic for the cells. Authors used cells derived from a mouse model of MSD to test the clearance capability of TFEB in this disease. They transfected MSD cells with a TFEB vector or an empty vector and monitored the accumulation of glycosaminoglycans 48 hours post-transfection. They found that TFEB was able to promote the clearance of stored glycosaminoglycans (FIG. 17) and to reverse the subsequent cellular vacuolization, as demonstrated by electron microscopy analysis (FIG. 18). Authors tested the clearance capability of TFEB on an additional model of lysosomal storage disorder, the Pompe disease, in which a defect in the acid alpha-glucosidase gene leads to the intralysosomal accumulation of glycogen and subsequent extensive vacuolization of the cell. Authors transfected human fibroblasts derived from a Pompe patient with a TFEB-3×FLAG vector and monitored the shape and the number of lysosomes in the cells. Cells transfected with TFEB-3×FLAG were found to diminish the amount of undigested glycogen, as demonstrated by the decreased number of lysosomal vesicles compared to non-transfected cells (FIG. 19). Together, these data indicate that the enhancement of the lysosomal activity by acting on the CLEAR network can provide in principle a polyvalent therapy against different lysosomal storage disorders.

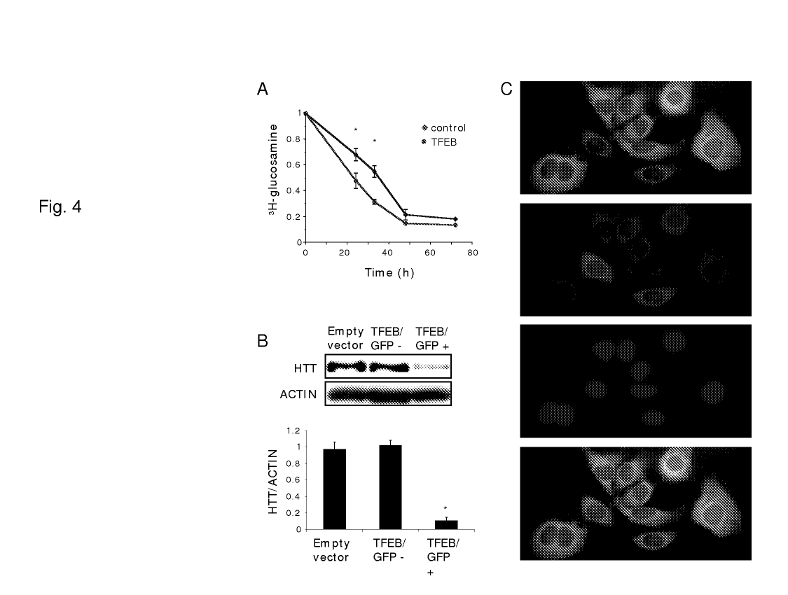

To test the ability of TFEB to enhance lysosome-dependent degradation pathways authors analyzed the degradation of glycosaminoglycans (GAGs) in a pulse-chase experiment. TFEB stable transfectants displayed a faster rate of GAG clearance compared to controls (FIG. 4A). Authors also investigated the ability of TFEB to induce the degradation of the polyglutamine (polyQ) expanded huntingtin protein responsible for Huntington disease using the rat striatal cell model HD43 that carries an inducible transgene for mutant huntingtin (19). Immunoblot analyses showed a strong decrease of mutant huntingtin in TFEB-overexpressing cells compared to controls (FIG. 4B). In a parallel experiment, induced HD43 cells were electroporated with a 3×FLAG-TFEB construct. Immunofluorescence analyses showed that the cells that are positive for 3×FLAG-TFEB show little, if any, huntingtin accumulation (FIG. 4C).

Authors have discovered a cellular program that regulates lysosomal biogenesis and participates in macromolecule clearance. Lysosomal enhancement as a cellular response to pathogenic accumulation has been observed in neurodegenerative diseases (20-22). Interestingly, cathepsin D (23, 24), one of the key enzymes involved in the degradation of neurotoxic proteins, belongs to the CLEAR network and is induced by TFEB overexpression. Of particular interest is also the observation that miR-128, which authors used for TFEB downregulation, is significantly up-regulated in the brain of patients with Alzheimer's disease (25) and in both prion- and chemical-induced neurodegeneration (26, 27). An appealing perspective would be the use of the CLEAR network as a therapeutic target to enhance cellular response to intracellular pathogenic accumulation in neurodegenerative diseases.

TABLE 1
Distribution of CLEAR elements in the promoters of human lysosomal genes.

| | | | | |
|---|---|---|---|---|
| Gene | | | | Seq Id |
| symbol | Gene name | CLEAR element | Position* | No. |

Membrane transporters

| | | | | |
|---|---|---|---|---|
| ABCA2 | ATP-binding cassette, sub-family A (ABC1), | GTCGCGTGAC | −187 | 1 |
| | member 2 | | | |
| ABCB9 | ATP-binding cassette, sub-family B (MDR/TAP), | CTCACCTGGT | 94 | 2 |
| | member 9 | | | |
| CLCN7 | chloride channel 7 | ATCACGTGGC | −103 | 3 |
| | | GTCACGTGGC | −83 | 4 |
| CLN3 | ceroid-lipofuscinosis, neuronal 3, juvenile | AGCACGTGAT | −24 | 5 |
| | | GTCACGTGAT | 6 | 6 |
| CLN5 | ceroid-lipofuscinosis, neuronal 5 | CTCAAGTGTG | 50 | 7 |
| | | TTCAGGTGCC | 74 | 8 |
| CTNS | cystinosis, nephropathic | GTCAGGTGGC | −32 | 9 |
| | | GTCAGGTGAC | −18 | 10 |
| LAPTM4A | lysosomal-associated protein transmembrane 4 | GTCACGTTAT | −372 | 11 |
| | alpha | | | |
| | | GTGACGCTTC | −356 | 12 |
| LMBRD1 | LMBR1 domain containing 1 | — | — | |
| MCOLN1 | mucolipin 1 | GTCACGTGAG | −47 | 13 |
| | | GTCACGTGAC | −20 | 14 |
| | | ATCAGCTGAT | 0 | 15 |
| MFSD8 | major facilitator superfamily domain containing 8 | GTCAGGTGCG | −15 | 16 |
| NPC1 | Niemann-Pick disease, type C1 | TTCAGGTGAC | −383 | 17 |
| SCARB2 | scavenger receptor class B, member 2 | CTCAGGCGCC | −134 | 18 |
| | | GGCACATGAC | −57 | 19 |
| SLC17A5 | solute carrier family 17 (anion/sugar | GCCAGGTGGC | 47 | 20 |
| | transporter), member 5 | | | |
| | | CTCACGTAGG | 68 | 21 |
| SLC36A1 | solute carrier family 36 (proton/amino acid | AGCACGTGAC | −44 | 22 |
| | symporter), member 1 | | | |
| | | ATCACGTGAT | −9 | 23 |

Hydrolases

| | | | | |
|---|---|---|---|---|
| ACP2 | acid phosphatase 2 | — | — | |
| ACP5 | acid phosphatase 5, tartrate resistant | CTCACCTGGG | 8 | 24 |
| AGA | aspartylglucosaminidase | — | — | |
| ARSA | arylsulfatase A | GCCAAGTGAC | 80 | 25 |
| ARSB | arylsulfatase B | [symbol] | 288 | 26 |
| ARSG | arylsulfatase G | GCCACGTGTG | 183 | 27 |
| ASAH1 | N-acylsphingosine amidohydrolase 1 | GTCACGCGGC | −41 | 28 |
| CPVL | carboxypeptidase, vitellogenic-like | GTCATGTGAG | −123 | 29 |
| CTBS | di-N-acetyl-chitobiase | — | — | |
| CTSA | cathepsin A | GTCACGTGGC | −50 | 30 |
| | | TTCACGTGAC | −33 | 31 |
| CTSB | cathepsin B | GTCACGTGGG | −7 | 32 |
| CTSC | cathepsin C | TTCACCTGAC | −343 | 33 |
| CTSD | cathepsin D | CCCACGTGAC | 16 | 34 |
| | | GTCAGCTGAT | 48 | 35 |
| CTSF | cathepsin F | CCCACGTGCC | −83 | 36 |
| CTSH | cathepsin H | CCCAGTTGAC | 30 | 37 |
| CTSK | cathepsin K | GTCACATGTG | −650 | 38 |
| | | TTCAAGTGCT | −615 | 39 |
| CTSL1 | cathepsin L1 | GTCAGGCGAA | 43 | 40 |
| CTSS | cathepsin S | CTCAAGTGAT | −66 | 41 |
| CTSZ | cathepsin Z | TTCAGGTGCC | −166 | 42 |
| DNASE2 | deoxyribonuclease II, lysosomal | GCCAGGTGCC | 63 | 43 |
| ENTPD4 | ectonucleoside triphosphate | — | — | |
| | diphosphohydrolase 4 | | | |
| FUCA1 | alpha-L fucosidase | — | — | |
| GAA | alpha-glucosidase | GTCACGTGAC | 20 | 44 |
| | | GTCACGTGAC | 65 | 45 |
| GALC | galactosylceramidase | GTCATGTGAC | 1 | 46 |
| GALNS | galactosamine (N-acetyl)-6-sulfate sulfatase | [symbol] | −147 | 47 |
| | | GTCACGCGGC | −128 | 48 |
| | | GTCACGTGGC | −5 | 49 |
| GBA | beta-glucosidase | GTCATGTGAC | −64 | 50 |
| | | ATCACATGAC | −44 | 51 |
| GGH | gamma-glutamyl hydrolase | CTCACGCGAG | −31 | 52 |
| GLA | alpha-galactosidase | CTCACGTAAG | −223 | 53 |
| | | ATCACGTGAG | −207 | 54 |
| | | GTCATGTGAG | −190 | 55 |
| | | GTCACGTGAG | −174 | 56 |
| GLB1 | beta-galactosidase | GTCACGCGGC | −139 | 57 |
| | | GTCAAGTGAC | −3 | 58 |
| GNS | glucosamine (N-acetyl)-6-sulfatase | GTCACGTGAC | −42 | 59 |
| | | CTCACGTGAT | −2 | 60 |
| GUSB | beta-glucuronidase | GTCACGCGAC | −49 | 61 |
| HEXA | beta-hexosaminidase subunit alpha | GTCACGTGAT | −3 | 62 |
| | | CTCACCTGAC | 33 | 63 |
| | | CTCACGTGGC | 49 | 64 |
| HEXB | beta-hexosaminidase subunit beta | GTCATCTGAC | 3 | 65 |
| HGSNAT | heparan-alpha-glucosaminide N- | — | — | |
| | acetyltransferase | | | |
| HPSE | Heparanase | GCCAGGTGAG | 84 | 66 |
| HYAL1 | hyaluronoglucosaminidase 1 | — | — | |
| HYAL2 | hyaluronoglucosaminidase 2 | GTCACCTGGC | −194 | 67 |
| IDS | Iduronate-2-sulfatase | — | — | |
| IDUA | alpha-L-iduronidase | GTCACATGGG | 1 | 68 |
| LGMN | legumain | — | — | |
| LIPA | acid lipase | ATCAGATGCC | 34 | 69 |
| LYPLA3 | lysophospholipase 3 | GTCACCTGAG | −431 | 70 |
| MAN2B1 | alpha-mannosidase, class 2B, member 1 | CTCCCGTGAG | −87 | 71 |
| MAN2B2 | alpha-mannosidase, class 2B, member 2 | — | — | |
| MANBA | beta-mannosidase | CTCAGCTGAC | −47 | 72 |
| NAAA | N-acylethanolamine acid amidase | — | — | |
| NAGA | alpha-N-acetylgalactosaminidase | CCTTCGTGAG | −23 | 73 |
| | | CTCACTGGAA | −5 | 74 |
| | | ATCAGGTTAC | 18 | 75 |
| | | GTCAGAAGCG | 37 | 76 |
| NAGLU | alpha-N-acetylglucosaminidase | [symbol] | 178 | 77 |
| NEU1 | sialidase 1 | GTCACGCGCT | −116 | 78 |
| | | GTCAGCTGAC | 69 | 79 |
| NEU4 | sialidase 4 | GTCATTTGAG | −336 | 80 |
| P76 | mannose-6-phosphate protein p76 | GTCACGTGAC | −12 | 81 |
| PPT1 | palmitoyl-protein thioesterase 1 | GTCATGTGAC | 39 | 82 |
| PPT2 | palmitoyl-protein thioesterase 2 | — | — | |
| RNASET2 | ribonuclease 6 | GGCAGGTGAG | −41 | 83 |
| SCPEP1 | serine carboxypeptidase 1 | GTCACGTGAT | −26 | 84 |
| SGSH | N-sulfoglucosamine sulfohydrolase | [symbol] | −85 | 85 |
| SIAE | sialic acid acetylesterase | — | — | |
| SMPD1 | sphingomyelin phosphodiesterase | ATCAGCTGTC | −14 | 86 |
| | | GTCAGCCGAC | 51 | 87 |
| TMEM55B | transmembrane protein 55B | AACACGTGAC | −288 | 88 |
| | | GTCACGTGCA | −193 | 89 |
| | | GTCATGTGAC | −154 | 90 |
| | | ATCACGTGCT | −36 | 91 |
| TPP1 | tripeptidyl peptidase I | CTCATGTGAT | −15 | 92 |
| | | GTCACATGAC | −3 | 93 |

Signaling

| | | | |
|---|---|---|---|
| CREG1 | cellular repressor of E1A-stimulated genes 1 | — | — |
| LITAF | lipopolysaccharide-induced TNF factor | — | — |
| TMEM9 | transmembrane protein 9 | — | — |

Other functions

| | | | | |
|---|---|---|---|---|
| CD63 | CD63 molecule | GTCACATGAG | 14 | 94 |
| CD68 | CD68 molecule | TCAACTGCCC | −82 | 95 |
| | | CCCATGTGAC | −55 | 96 |
| GM2A | GM2 ganglioside activator | — | — | |
| IFI30 | interferon, gamma-inducible protein 30 | CTCACGTGCC | −174 | 97 |
| LAMP1 | lysosomal-associated membrane protein 1 | GTCACGTGGG | −196 | 98 |
| | | GTCACGTGCC | −180 | 99 |
| | | GTCACGTGCC | −163 | 100 |
| | | GTCACGTGTC | −146 | 101 |
| | | ATCACGTGAC | −32 | 102 |
| | | CTCACGTGAC | −5 | 103 |
| LAMP2 | lysosomal-associated membrane protein 2 | — | — | |
| LAMP3 | lysosomal-associated membrane protein 3 | — | — | |
| MPO | myeloperoxidase | ATCAGGTGAG | 7 | 104 |
| NCSTN | nicastrin | — | — | |
| NPC2 | Niemann-Pick disease, type C2 | CTCAGCTGTG | −19 | 105 |
| | | GTCGCCTGAC | 5 | 106 |
| | | GTCTTGTGAC | 49 | 107 |
| OSTM1 | osteopetrosis associated transmembrane | — | — | |
| | protein 1 | | | |
| PCYOX1 | prenylcysteine oxidase 1 | — | — | |
| PSAP | prosaposin | ATCAGCTGAC | 5 | 108 |
| TMEM74 | transmembrane protein 74 | — | — | |

Unknown function

| | | | | |
|---|---|---|---|---|
| C2orf18 | chromosome 2 open reading frame 18 | GTCACGTGAC | −33 | 109 |
| C7orf28A | chromosome 7 open reading frame 28A | — | — | |
| EPDR1 | ependymin related protein 1 | — | — | |
| LAPTM5 | lysosomal-associated multispanning membrane | — | — | |
| | protein 5 | | | |
| TMEM92 | transmembrane protein 92 | — | — | |

*Position refers to the transcription start site

TABLE 2
Gene Ontology (GO) analysis of CLEAR genes.

| | Gene | Fold | |
|---|---|---|---|
| GO Term | Count | enr. | P value |
| Cellular Compartment | | | |
| GO: 0005764~lysosome | 23 | 7.2 | 1.03E−12 |
| GO: 0016471~vacuolar proton-transporting | 3 | 37.3 | 2.48E−03 |
| V-type ATPase complex | | | |
| GO: 0005768~endosome | 10 | 3.2 | 4.34E−03 |
| Biological Process | | | |
| GO: 0007040~lysosome organization and | 7 | 24.3 | 2.56E−07 |
| biogenesis | | | |
| GO: 0016192~vesicle-mediated transport | 20 | 2.6 | 2.73E−04 |
| GO: 0032940~secretion by cell | 13 | 3 | 1.43E−03 |
| GO: 0006643~membrane lipid metabolic | 11 | 3.4 | 1.56E−03 |
| process | | | |
| GO: 0046034~ATP metabolic process | 6 | 6.7 | 1.94E−03 |
| GO: 0006644~phospholipid metabolic | 9 | 3.7 | 3.12E−03 |
| process | | | |
| GO: 0045045~secretory pathway | 11 | 3 | 3.49E−03 |
| Molecular Function | | | |
| GO: 0016787~hydrolase activity | 58 | 1.6 | 2.39E−04 |
| GO: 0016298~lipase activity | 8 | 5.3 | 7.98E−04 |
| GO: 0016798~hydrolase activity, acting | 9 | 4.2 | 1.37E−03 |
| on glycosyl bonds | | | |
| GO: 0016805~dipeptidase activity | 3 | 20.2 | 9.07E−03 |

TABLE 3
Genes differentially expressed following TFEB transient overexpression.

| | | | Fold |
|---|---|---|---|
| Gene Symbol | Protein | Process | change |
| ATP6V0D2 | ATPase, H+ transporting, lysosomal 38kDa, V0 | Lysosomal | 2908 |
| | subunit d2 | acidification | |
| RASGRP3 | RAS guanyl releasing protein 3 (calcium and DAG- | Signal transduction | 92.8 |
| | regulated) | | |
| ZNF57 | zinc finger protein 57 | unknown | 60.7 |
| TRIM63 | tripartite motif-containing 63 | Protein degradation | 40.6 |
| SLC16A6 | solute carrier family 16, member 6 (monocarboxylic | Drug disposition | 38.5 |
| | acid transporter 7) | | |
| PER3 | period homolog 3 (Drosophila) | Circadian rhythms | 37.7 |
| TM4SF19 | transmembrane 4 L six family member 19 | unknown | 23.6 |
| CPA2 | carboxypeptidase A2 (pancreatic) | Protein degradation | 19.4 |
| C1orf54 | chromosome 1 open reading frame 54 | unknown | 17.2 |
| SULT1C2 | sulfotransferase family, cytosolic, 1C, member 2 | Sulfate conjugation | 13.9 |
| CTNS | cystinosis, nephropathic | Lysosomal carrier | 13.6 |
| NR1D1 | nuclear receptor subfamily 1, group D, member 1 | Circadian rhythms | 12.5 |
| UCA1 | urothelial cancer associated 1 | unknown | 12.3 |
| UPP1 | uridine phosphorylase 1 | Catabolism of | 11.1 |
| | | nucleotides | |
| SLC19A2 | solute carrier family 19 (thiamine transporter), | Thiamin transport | 10.3 |
| | member 2 | | |
| GPR56 | G protein-coupled receptor 56 | Signal transduction | 9.8 |
| SLAMF7 | SLAM family member 7 | Immune response | 9.6 |
| PRKAG2 | protein kinase, AMP-activated, gamma 2 non- | Energy metabolism | 8.6 |
| | catalytic subunit | | |
| STS | steroid sulfatase (microsomal), isozyme S | Microsomal hydrolase | 8.4 |
| CCRL2 | similar to chemokine (C-C motif) receptor-like 2 | Immune response | 8.3 |
| MAP3K13 | mitogen-activated protein kinase kinase kinase 13 | Signal transduction | 7.8 |
| GIPR | gastric inhibitory polypeptide receptor | Insulin metabolism | 7.6 |
| SEMA3D | sema domain, immunoglobulin domain (Ig), short | Signal transduction | 7.4 |
| | basic domain, secreted, (semaphorin) 3D | | |
| ANKRD1 | ankyrin repeat domain 1 (cardiac muscle) | Signal transduction | 7.2 |
| BHLHB3 | basic helix-loop-helix domain containing, class B, 3 | Circadian rhythms | 6.8 |
| VASN | vasorin | Signal transduction | 6.5 |
| PTP4A3 | protein tyrosine phosphatase type IVA, member 3 | Cell growth | 6.4 |
| FNIP2 | folliculin interacting protein 2 | unknown | 6.3 |
| PLK3 | polo-like kinase 3 (Drosophila) | Protein | 6.2 |
| | | phosphorylation | |
| CPA4 | carboxypeptidase A4 | Protein degradation | 6.1 |
| ST3GAL1 | ST3 beta-galactoside alpha-2,3-sialyltransferase 1 | Protein glycosylation | 6.1 |
| CSF1R | colony stimulating factor 1 receptor, formerly | Immune response | 5.8 |
| | McDonough feline sarcoma viral (v-fms) oncogene | | |
| | homolog | | |
| SUV39H1 | suppressor of variegation 3-9 homolog 1 | Chromatin | 5.7 |
| | (Drosophila) | modification | |
| ZDHHC3 | zinc finger, DHHC-type containing 3 | unknown | 5.5 |
| IL6R | interleukin 6 receptor | Immune response | 5.5 |
| FAM27E3 | family with sequence similarity 27, member E3 | unknown | 5.5 |
| C1R | complement component 1, r subcomponent | Immune response | 5.5 |
| FAM102A | family with sequence similarity 102, member A | unknown | 5.4 |
| SECTM1 | secreted and transmembrane 1 | Immune response | 5.4 |
| FAM124A | family with sequence similarity 124A | unknown | 5.3 |
| RGS16 | regulator of G-protein signaling 16 | Signal transduction | 5.3 |
| RASD2 | RASD family, member 2 | Signal transduction | 5.3 |
| PLCXD1 | phosphatidylinositol-specific phospholipase C, X | unknown | 5.2 |
| | domain containing 1 | | |
| AHNAK2 | AHNAK nucleoprotein 2 | unknown | 5.1 |
| ASAH1 | N-acylsphingosine amidohydrolase (acid | Lysosomal hydrolase | 5.1 |
| | ceramidase) 1 | | |
| SLC26A11 | solute carrier family 26, member 11 | Sulfate transport | 5.1 |
| TMEM80 | transmembrane protein 80 | unknown | 5.1 |
| HEXA | hexosaminidase A (alpha polypeptide) | Lysosomal hydrolase | 5.1 |
| SLC26A9 | solute carrier family 26, member 9 | Sulfate transport | 5.0 |
| TGM5 | transglutaminase 5 | Epidermis | 5.0 |
| | | development | |
| MCOLN1 | mucolipin 1 | Lysosomal carrier | 5.0 |
| FLJ41484 | hypothetical LOC650669 | unknown | 5.0 |
| ALOXE3 | arachidonate lipoxygenase 3 | Inflammatory | 4.9 |
| | | response | |
| CHKA | choline kinase alpha | Lipid metabolism | 4.9 |
| C17orf80 | chromosome 17 open reading frame 80 | unknown | 4.7 |
| LIF | leukemia inhibitory factor (cholinergic differentiation | Immune response | 4.6 |
| | factor) | | |
| ADFP | adipose differentiation-related protein | Adipocyte | 4.6 |
| | | differentiation | |
| SLC20A1 | solute carrier family 20 (phosphate transporter), | Sulfate transport | 4.6 |
| | member 1 | | |
| DKFZp451A211 | DKFZp451A211 protein | unknown | 4.6 |
| ATP6V0D1 | ATPase, H+ transporting, lysosomal 38kDa, V0 | Lysosomal | 4.5 |
| | subunit d1 | acidification | |
| DEXI | dexamethasone-induced transcript | unknown | 4.4 |
| FAM21B | family with sequence similarity 21, member B | unknown | 4.4 |
| PLEKHM1 | pleckstrin homology domain containing, family M | Lysosomal | 4.4 |
| | (with RUN domain) member 1 | metabolism | |
| CEP72 | centrosomal protein 72kDa | Centrosome | 4.3 |
| | | component | |
| DVL2 | dishevelled, dsh homolog 2 (Drosophila) | Signal transduction | 4.3 |
| SNAI2 | snail homolog 2 (Drosophila) | Development | 4.3 |
| LSS | lanosterol synthase (2,3-oxidosqualene-lanosterol | Cholesterol | 4.2 |
| | cyclase) | metabolism | |
| HSPC159 | galectin-related protein | unknown | 4.2 |
| RAET1E | retinoic acid early transcript 1E | Immune response | 4.2 |
| TCTEX1D2 | Tctex1 domain containing 2 | unknown | 4.2 |
| SERTAD2 | SERTA domain containing 2 | Cell proliferation | 4.2 |
| LOC201164 | similar to CG12314 gene product | unknown | 4.1 |
| TMEFF1 | transmembrane protein with EGF-like and two | Signal transduction | 4.1 |
| | follistatin-like domains 1 | | |
| VPS18 | vacuolar protein sorting 18 homolog (S. cerevisiae) | Lysosomal trafficking | 4.1 |
| SYNJ2 | synaptojanin 2 | Metabolism | 4.1 |
| LOC100132929 | similar to hCG24378 | unknown | 4.1 |
| HLA-B | major histocompatibility complex, class I, B | Proteasome | 4.1 |
| | | degradation | |
| CRYAB | crystallin, alpha B | Apoptosis | 4.1 |
| CABLES1 | Cdk5 and Abl enzyme substrate 1 | Cell proliferation and | 4.0 |
| | | differentiation | |
| GRN | granulin | Inflammatory | 4.0 |
| | | response | |
| UVRAG | UV radiation resistance associated gene | Autophagy | 4.0 |
| CAMKK1 | calcium/calmodulin-dependent protein kinase kinase | Immune response | 4.0 |
| | 1, alpha | | |
| SPINK1 | serine peptidase inhibitor, Kazal type 1 | Protease inhibitor | 4.0 |
| CLEC17A | C-type lectin and transmembrane domain- | unknown | 4.0 |
| | containing protein FLJ45910 | | |
| PPARGC1A | peroxisome proliferator-activated receptor gamma, | Energy metabolism | 3.9 |
| | coactivator 1 alpha | | |
| TPP1 | tripeptidyl peptidase I | Lysosomal hydrolase | 3.9 |
| SFXN3 | sideroflexin 3 | Mitochondrial carrier | 3.9 |
| HES1 | hairy and enhancer of split 1, (Drosophila) | Development | 3.9 |
| EIF2C4 | eukaryotic translation initiation factor 2C, 4 | Gene silencing | 3.9 |
| VPS11 | vacuolar protein sorting 11 homolog (S. cerevisiae) | Lysosomal trafficking | 3.9 |
| CTSF | cathepsin F | Lysosomal hydrolase | 3.9 |
| KCNAB2 | potassium voltage-gated channel, shaker-related | unknown | 3.8 |
| | subfamily, beta member 2 | | |
| SETDB2 | SET domain, bifurcated 2 | Chromatin | 3.8 |
| | | modification | |
| PSG4 | pregnancy specific beta-1-glycoprotein 4 | Defense response | 3.8 |
| C12orf49 | chromosome 12 open reading frame 49 | unknown | 3.8 |
| BLVRB | biliverdin reductase B (flavin reductase (NADPH)) | Metabolism | 3.8 |
| APBB3 | amyloid beta (A4) precursor protein-binding, family | APP metabolism | 3.8 |
| | B, member 3 | | |
| UCK1 | uridine-cytidine kinase 1 | Metabolism | 3.7 |
| HSPB8 | heat shock 22kDa protein 8 | Cell proliferation | 3.7 |
| LRRC8B | leucine rich repeat containing 8 family, member B | unknown | 3.7 |
| NHEDC2 | Na+/H+ exchanger domain containing 2 | Mitochondrial carrier | 3.7 |
| TIAF1 | TGFB1-induced anti-apoptotic factor 1 | Apoptosis | 3.7 |
| FAM21A | family with sequence similarity 21, member A | unknown | 3.7 |
| STOM | stomatin | unknown | 3.7 |
| HEY1 | hairy/enhancer-of-split related with YRPW motif 1 | Development | 3.6 |
| BHLHB2 | basic helix-loop-helix domain containing, class B, 2 | Development | 3.6 |
| NUP50 | nucleoporin 50kDa | Nuclear pore | 3.6 |
| | | component | |
| WDR81 | WD repeat domain 81 | unknown | 3.6 |
| ACBD3 | acyl-Coenzyme A binding domain containing 3 | Golgi transport | 3.6 |
| FBXO32 | F-box protein 32 | Ubiquitylation | 3.6 |
| GEM | GTP binding protein overexpressed in skeletal | Signal transduction | 3.6 |
| | muscle | | |
| UGDH | UDP-glucose dehydrogenase | Biosiynthesis of | 3.6 |
| | | GAGs | |
| HOXB9 | homeobox B9 | Cell proliferation and | 3.6 |
| | | differentiation | |
| LOC100128975 | similar to Zinc finger protein 626 | unknown | 3.6 |
| LYPD5 | LY6/PLAUR domain containing 5 | Signal transduction | 3.6 |
| CLC | Charcot-Leyden crystal protein | Lipid metabolism | 3.6 |
| CD22 | CD22 molecule | Immune response | 3.5 |
| NIT1 | nitrilase 1 | Metabolism | 3.5 |
| SRRD | SRR1 domain containing | unknown | 3.5 |
| VEGFA | vascular endothelial growth factor A | Development | 3.5 |
| MMP12 | matrix metallopeptidase 12 (macrophage elastase) | Protein degradation | 3.5 |
| LAMA1 | laminin, alpha 1 | Cell proliferation and | 3.5 |
| | | differentiation | |
| HMOX1 | heme oxygenase (decycling) 1 | Metabolism | 3.5 |
| SLC25A16 | solute carrier family 25 (mitochondrial carrier; | Mitochondrial carrier | 3.5 |
| | Graves disease autoantigen), member 16 | | |
| KIAA1632 | KIAA1632 | unknown | 3.5 |
| HK2 | hexokinase 2 | Energy metabolism | 3.5 |
| KIFC3 | kinesin family member C3 | Golgi organization | 3.5 |
| | | and biogenesis | |
| CD68 | CD68 molecule | Lysosomal | 3.5 |
| | | metabolism | |
| CHUK | conserved helix-loop-helix ubiquitous kinase | Immune response | 3.5 |
| RAB17 | Ras-related protein Rab-17 | Signal transduction | 3.5 |
| CXCL16 | chemokine (C-X-C motif) ligand 16 | Immune response | 3.5 |
| KIAA1737 | KIAA1737 | unknown | 3.4 |
| CRY1 | cryptochrome 1 (photolyase-like) | Circadian rhythms | 3.4 |
| NDRG1 | N-myc downstream regulated gene 1 | Cell proliferation and | 3.4 |
| | | differentiation | |
| NEDD4L | neural precursor cell expressed, developmentally | Ubiquitylation | 3.4 |
| | down-regulated 4-like | | |
| KCNN4 | potassium intermediate/small conductance calcium- | Defense response | 3.4 |
| | activated channel, subfamily N, member 4 | | |
| NAGK | N-acetylglucosamine kinase | Metabolism | 3.4 |
| FAM54A | family with sequence similarity 54, member A | unknown | 3.4 |
| PSEN2 | presenilin 2 (Alzheimer disease 4) | APP metabolism | 3.4 |
| PPIF | peptidylprolyl isomerase F (cyclophilin F) | Mitochondrial | 3.4 |
| | | metabolism | |
| LOC654433 | hypothetical LOC654433 | unknown | 3.4 |
| DCPS | decapping enzyme, scavenger | mRNA metabolism | 3.4 |
| PDXDC2 | pyridoxal-dependent decarboxylase domain | Metabolism | 3.4 |
| | containing 2 | | |
| PLCD1 | phospholipase C, delta 1 | Phospholipid | 3.4 |
| | | metabolic process | |
| STK19 | serine/threonine kinase 19 | unknown | 3.4 |
| LCN8 | lipocalin 8 | Metabolism | 3.4 |
| DUSP10 | dual specificity phosphatase 10 | Signal transduction | 3.3 |
| SBNO2 | strawberry notch homolog 2 (Drosophila) | Immune response | 3.3 |
| LY6K | lymphocyte antigen 6 complex, locus K | unknown | 3.3 |
| GSTO1 | glutathione S-transferase omega 1 | Metabolism | 3.3 |
| SLC29A1 | solute carrier family 29 (nucleoside transporters), | Metabolism | 3.3 |
| | member 1 | | |
| CD300C | CD300c molecule | Immune response | 3.3 |
| AVPI1 | arginine vasopressin-induced 1 | unknown | 3.3 |
| DAB2 | disabled homolog 2, mitogen-responsive | Lysosomal trafficking | 3.3 |
| | phosphoprotein (Drosophila) | | |
| SLCO4A1 | solute carrier organic anion transporter family, | unknown | 3.3 |
| | member 4A1 | | |
| GSR | glutathione reductase | Metabolism | 3.3 |
| UST | uronyl-2-sulfotransferase | Metabolism | 3.3 |
| PTTG1IP | pituitary tumor-transforming 1 interacting protein | Signal transduction | 3.3 |
| ICAM1 | intercellular adhesion molecule 1 (CD54), human | Immune response | 3.3 |
| | rhinovirus receptor | | |
| NUFIP1 | nuclear fragile X mental retardation protein | Transcription | 3.3 |
| | interacting protein 1 | | |
| RAB3IL1 | RAB3A interacting protein (rabin3)-like 1 | Exocytosis | 3.3 |
| TEAD3 | TEA domain family member 3 | Pregnancy | 3.2 |
| GDF15 | growth differentiation factor 15 | Signal transduction | 3.2 |
| PIM1 | pim-1 oncogene | Cell proliferation | 3.2 |
| TAF4B | TAF4b RNA polymerase II, TATA box binding | Transcription | 3.2 |
| | protein (TBP)-associated factor, 105kDa | | |
| MFSD1 | major facilitator superfamily domain containing 1 | unknown | 3.2 |
| CTSB | cathepsin B | Lysosomal hydrolase | 3.2 |
| EPS15L1 | epidermal growth factor receptor pathway substrate | Endocytosis | 3.2 |
| | 15-like 1 | | |
| SPTBN1 | spectrin, beta, non-erythrocytic 1 | Cytoskeleton | 3.2 |
| | | component | |
| CSTB | cystatin B (stefin B) | Protease inhibitor | 3.2 |
| HKDC1 | hexokinase domain containing 1 | Energy metabolism | 3.2 |
| LPAR5 | lysophosphatidic acid receptor 5 | Signal transduction | 3.2 |
| CTSD | cathepsin D | Lysosomal hydrolase | 3.2 |
| LINS1 | lines homolog 1 (Drosophila) | unknown | 3.2 |
| IGF2R | insulin-like growth factor 2 receptor | Lysosomal trafficking | 3.2 |
| RCSD1 | RCSD domain containing 1 | unknown | 3.2 |
| CSPG4 | chondroitin sulfate proteoglycan 4 | Signal transduction | 3.2 |
| VAC14 | Vac14 homolog (S. cerevisiae) | Signal transduction | 3.2 |
| CHRM4 | cholinergic receptor, muscarinic 4 | Signal transduction | 3.2 |
| IL16 | interleukin 16 (lymphocyte chemoattractant factor) | Immune response | 3.2 |
| SLC25A40 | solute carrier family 25, member 40 | Mitochondrial carrier | 3.2 |
| MTMR10 | myotubularin related protein 10 | Signal transduction | 3.2 |
| RLTPR | RGD motif, leucine rich repeats, tropomodulin | unknown | 3.2 |
| | domain and proline-rich containing | | |
| SH3RF2 | SH3 domain containing ring finger 2 | Ubiquitylation | 3.1 |
| PFKFB3 | 6-phosphofructo-2-kinase/fructose-2,6- | Energy metabolism | 3.1 |
| | biphosphatase 3 | | |
| TMEM16B | transmembrane protein 16B | unknown | 3.1 |
| DENND2D | DENN/MADD domain containing 2D | unknown | 3.1 |
| ADM | adrenomedullin | Signal transduction | 3.1 |
| SLC25A25 | solute carrier family 25 (mitochondrial carrier; | Mitochondrial carrier | 3.1 |
| | phosphate carrier), member 25 | | |
| SLC2A1 | solute carrier family 2 (facilitated glucose | Glucose transporter | 3.1 |
| | transporter), member 1 | | |
| ATP6V0B | ATPase, H+ transporting, lysosomal 21kDa, V0 | Lysosomal | 3.1 |
| | subunit b | acidification | |
| TOM1 | target of myb1 (chicken) | Endocytic trafficking | 3.1 |
| DDI2 | DDI1, DNA-damage inducible 1, homolog 2 (S. | Protein degradation | 3.1 |
| | cerevisiae) | | |
| SLC25A22 | solute carrier family 25 (mitochondrial carrier: | Mitochondrial carrier | 3.1 |
| | glutamate), member 22 | | |
| NAPA | N-ethylmaleimide-sensitive factor attachment | ER-Golgi transport | 3.1 |
| | protein, alpha | | |
| ESCO1 | Establishment of cohesion 1 homolog 1 (S. | DNA metabolism | 3.1 |
| | cerevisiae) | | |
| SETD4 | SET domain containing 4 | unknown | 3.1 |
| RRAGC | Ras-related GTP binding C | Autophagy | 3.1 |
| ATP6V1C1 | ATPase, H+ transporting, lysosomal 42kDa, V1 | Lysosomal | 3.1 |
| | subunit C1 | acidification | |
| PDP2 | pyruvate dehydrogenase phosphatase isoenzyme 2 | Mitochondrial | 3.1 |
| | | metabolism | |
| HSPBAP1 | HSPB (heat shock 27kDa) associated protein 1 | unknown | 3.1 |
| SUNC1 | Sad1 and UNC84 domain containing 1 | unknown | 3.1 |
| ITPKB | inositol 1,4,5-trisphosphate 3-kinase B | Signal transduction | 3.1 |
| RPP25 | ribonuclease P/MRP 25kDa subunit | RNA metabolism | 3.0 |
| CEP250 | centrosomal protein 250kDa | Centrosome | 3.0 |
| | | component | |
| TACC2 | transforming, acidic coiled-coil containing protein 2 | Centrosome | 3.0 |
| | | component | |
| FAM83G | family with sequence similarity 83, member G | unknown | 3.0 |
| ATP6V1B2 | ATPase, H+ transporting, lysosomal 56/58kDa, V1 | Lysosomal | 3.0 |
| | subunit B2 | acidification | |
| PDE2A | phosphodiesterase 2A, cGMP-stimulated | Signal transduction | 3.0 |
| NSMCE2 | non-SMC element 2, MMS21 homolog ((S. | DNA metabolism | 3.0 |
| | cerevisiae) | | |
| WBP2 | WW domain binding protein 2 | Signal transduction | 3.0 |
| ATP6V0A1 | ATPase, H+ transporting, lysosomal V0 subunit a1 | Lysosomal | 3.0 |
| | | acidification | |
| LYPD3 | LY6/PLAUR domain containing 3 | unknown | 3.0 |
| CTSA | cathepsin A | Lysosomal hydrolase | 3.0 |
| MCCC1 | methylcrotonoyl-Coenzyme A carboxylase 1 (alpha) | Metabolism | 3.0 |
| ATP6V1H | ATPase, H+ transporting, lysosomal 50/57kDa, V1 | Lysosomal | 3.0 |
| | subunit H | acidification | |
| NR1D2 | nuclear receptor subfamily 1, group D, member 2 | Circadian rhythms | 3.0 |
| CLCN7 | chloride channel 7 | Lysosomal | 3.0 |
| | | acidification | |
| RYBP | RING1 and YY1 binding protein | Transcription | 3.0 |
| LOC643338 | hypothetical LOC643338 | unknown | 3.0 |
| CLCN6 | chloride channel 6 | Endosomal | 3.0 |
| | | component | |
| ZSCAN5A | zinc finger and SCAN domain containing 5 | Transcription | 3.0 |
| FOLR1 | folate receptor 1 (adult) | Metabolism | 3.0 |
| TRAF5 | TNF receptor-associated factor 5 | Apoptosis | 3.0 |
| HIF1A | hypoxia-inducible factor 1, alpha subunit (basic | Transcription | 3.0 |
| | helix-loop-helix transcription factor) | | |
| PPP1R13B | protein phosphatase 1, regulatory (inhibitor) subunit | Apoptosis | 3.0 |
| | 13B | | |
| GBA | glucosidase, beta; acid (includes | Lysosomal hydrolase | 3.0 |
| | glucosylceramidase) | | |
| ELOVL7 | ELOVL family member 7, elongation of long chain | Metabolism | 3.0 |
| | fatty acids (yeast) | | |
| TRPM7 | transient receptor potential cation channel, | Calcium ion transport | 3.0 |
| | subfamily M, member 7 | | |
| GLA | galactosidase, alpha | Lysosomal hydrolase | 2.9 |
| MAFF | v-maf musculoaponeurotic fibrosarcoma oncogene | Inflammatory | 2.9 |
| | homolog F (avian) | response | |
| UAP1L1 | UDP-N-acteylglucosamine pyrophosphorylase 1-like | Metabolism | 2.9 |
| | 1 | | |
| ZNF330 | zinc finger protein 330 | unknown | 2.9 |
| PIP4K2C | phosphatidylinositol-5-phosphate 4-kinase, type II, | unknown | 2.9 |
| | gamma | | |
| FNBP1L | formin binding protein 1-like | Endocytosis | 2.9 |
| TNFAIP3 | tumor necrosis factor, alpha-induced protein 3 | Signal transduction | 2.9 |
| EPS8 | epidermal growth factor receptor pathway substrate | Signal transduction | 2.9 |
| | 8 | | |
| PTGES | prostaglandin E synthase | Signal transduction | 2.9 |
| SCPEP1 | serine carboxypeptidase 1 | Lysosomal hydrolase | 2.9 |
| GTF2H1 | general transcription factor IIH, polypeptide 1, | Transcription | 2.9 |
| | 62kDa | | |
| INSIG1 | insulin induced gene 1 | Cholesterol | 2.9 |
| | | metabolism | |
| ARAP3 | ArfGAP with RhoGAP domain, ankyrin repeat and | Cytoskeleton | 2.9 |
| | PH domain 3 | component | |
| TBC1D14 | TBC1 domain family, member 14 | Signal transduction | 2.9 |
| KCNK9 | potassium channel, subfamily K, member 9 | Potassium ion | 2.9 |
| | | transport | |
| TMCC3 | transmembrane and coiled-coil domain family 3 | unknown | 2.9 |
| AMPD3 | adenosine monophosphate deaminase (isoform E) | Metabolism | 2.9 |
| NAGPA | N-acetylglucosamine-1-phosphodiester alpha-N- | Lysosomal trafficking | 2.9 |
| | acetylglucosaminidase | | |
| GNS | glucosamine (N-acetyl)-6-sulfatase (Sanfilippo | Lysosomal hydrolase | 2.9 |
| | disease IIID) | | |
| TMEM38B | transmembrane protein 38B | Potassium ion | 2.9 |
| | | transport | |
| SH3BP2 | SH3-domain binding protein 2 | Signal transduction | 2.9 |
| PMP22 | peripheral myelin protein 22 | Myelin component | 2.9 |
| TOB1 | transducer of ERBB2, 1 | Cell proliferation | 2.9 |
| GRAMD1B | GRAM domain containing 1B | unknown | 2.8 |
| ST3GAL4 | ST3 beta-galactoside alpha-2,3-sialyltransferase 4 | Golgi metabolism | 2.8 |
| NEU1 | sialidase 1 (lysosomal sialidase) | Lysosomal hydrolase | 2.8 |
| GNPDA1 | glucosamine-6-phosphate deaminase 1 | Golgi metabolism | 2.8 |
| TMEM55B | transmembrane protein 55B | Lysosomal | 2.8 |
| | | component | |
| BRI3 | brain protein I3 | Cell differentiation | 2.8 |
| C5orf24 | hypothetical LOC134553 | unknown | 2.8 |
| CYB5R1 | cytochrome b5 reductase 1 | Metabolism | 2.8 |
| TMEM159 | transmembrane protein 159 | unknown | 2.8 |
| GGA2 | golgi associated, gamma adaptin ear containing, | Golgi metabolism | 2.8 |
| | ARF binding protein 2 | | |
| RREB1 | ras responsive element binding protein 1 | Transcription | 2.8 |
| TRAPPC2L | trafficking protein particle complex 2-like | ER-Golgi transport | 2.8 |
| PCGF1 | polycomb group ring finger 1 | Transcription | 2.8 |
| STK17B | serine/threonine kinase 17b | Apoptosis | 2.8 |
| MPHOSPH10 | M-phase phosphoprotein 10 (U3 small nucleolar | Ribosome biogenesis | 2.8 |
| | ribonucleoprotein) | | |
| LOC440957 | hypothetical LOC440957 | unknown | 2.8 |
| CFB | complement factor B | Immune response | 2.8 |
| HTRA2 | HtrA serine peptidase 2 | Apoptosis | 2.8 |
| JPH1 | junctophilin 1 | unknown | 2.8 |
| SPG21 | spastic paraplegia 21 (autosomal recessive, Mast | Signal transduction | 2.8 |
| | syndrome) | | |
| CCDC43 | coiled-coil domain containing 43 | unknown | 2.8 |
| ZCCHC8 | zinc finger, CCHC domain containing 8 | RNA metabolism | 2.7 |
| RAD9A | RAD9 homolog A (S. pombe) | DNA metabolism | 2.7 |
| GPR175 | G protein-coupled receptor 175 | Signal transduction | 2.7 |
| SNX8 | sorting nexin 8 | Transport | 2.7 |
| WDTC1 | WD and tetratricopeptide repeats 1 | unknown | 2.7 |
| AXUD1 | AXIN1 up-regulated 1 | unknown | 2.7 |
| PEA15 | phosphoprotein enriched in astrocytes 15 | Apoptosis | 2.7 |
| CD63 | CD63 molecule | Lysosomal | 2.7 |
| | | metabolism | |
| SPNS1 | spinster homolog 1 (Drosophila) | unknown | 2.7 |
| LAMP1 | lysosomal-associated membrane protein 1 | Lysosomal | 2.7 |
| | | metabolism | |
| C7orf20 | chromosome 7 open reading frame 20 | unknown | 2.7 |
| LAMB3 | laminin, beta 3 | Cell adhesion | 2.7 |
| PSAP | prosaposin (variant Gaucher disease and variant | Lysosomal hydrolase | 2.7 |
| | metachromatic leukodystrophy) | | |
| SNX27 | sorting nexin family member 27 | Endocytic trafficking | 2.7 |
| WIPI1 | WD repeat domain, phosphoinositide interacting 1 | Autophagy | 2.7 |
| ATP6V1E1 | ATPase, H+ transporting, lysosomal 31kDa, V1 | Lysosomal | 2.6 |
| | subunit E1 | acidification | |
| CDKN1A | cyclin-dependent kinase inhibitor 1A (p21, Cip1) | Cell cycle | 2.6 |
| C1orf85 | chromosome 1 open reading frame 85 | unknown | 2.6 |
| XRCC2 | X-ray repair complementing defective repair in | DNA metabolism | 0.4 |
| | Chinese hamster cells 2 | | |
| DDX58 | DEAD (Asp-Glu-Ala-Asp) box polypeptide 58 | Immune response | 0.4 |
| BIRC3 | baculoviral IAP repeat-containing 3 | Apoptosis | 0.4 |
| DNAJB4 | DnaJ (Hsp40) homolog, subfamily B, member 4 | Stress response | 0.3 |
| LOC644714 | hypothetical protein LOC644714 | unknown | 0.3 |
| LCE2C | late cornified envelope 2C | Keratinization | 0.3 |
| LOC646993 | similar to high-mobility group box 3 | unknown | 0.2 |

TABLE 4
Gene Ontology (GO) terms enriched within the set of genes
upregulated following TFEB transient overexpression.

| | Gene | Fold | |
|---|---|---|---|
| GO Term | Count | enr. | P-value |
| Cellular Compartment | | | |
| GO: 0005764~lysosome | 17 | 8.2 | 2.50E−10 |
| GO: 0005765~lysosomal membrane | 8 | 15.4 | 7.65E−07 |
| GO: 0005768~endosome | 7 | 4.9 | 2.04E−04 |
| GO: 0048770~pigment granule | 10 | 8.0 | 2.33E−04 |
| GO: 0042470~melanosome | 7 | 8.0 | 2.33E−04 |
| Biological Process | | | |
| GO: 0015992~proton transport | 7 | 7.0 | 4.87E−04 |
| Molecular Function | | | |
| GO: 0022857~transmembrane transporter | 27 | 2.5 | 1.78E−05 |
| activity | | | |
| GO: 0003824~catalytic activity | 86 | 1.4 | 1.80E−04 |
| GO: 0019829~cation-transporting ATPase | 6 | 10.3 | 2.75E−04 |
| activity | | | |

TABLE 5
Sequences of oligos used in real-time qPCR analysis

| | | |
|---|---|---|
| Gene name | Forward primer | Reverse primer |

Expression analysis

| | | |
|---|---|---|
| TFEB | CCAGAAGCGAGAGCTCACAGAT | TGTGATTGTCTTTCTTCTGCCG |
| | Seq Id No. 114 | Seq Id No. 115 |
| ARSA | AGAGCTTTGCAGAGCGTTCAG | ATACGCATGGTCTCAGGTCCA |
| | Seq Id No. 116 | Seq Id No. 117 |
| ARSB | ATCAGTGAAGGAAGCCCATCC | ACACGGTGAAGAGTCCACGAA |
| | Seq Id No. 118 | Seq Id No. 119 |
| ATP6V0E1 | CATTGTGATGAGCGTGTTCTGG | AACTCCCCGGTTAGGACCCTTA |
| | Seq Id No. 120 | Seq Id No. 121 |
| ATP6V1H | GGAAGTGTCAGATGATCCCCA | CCGTTTGCCTCGTGGATAAT |
| | Seq Id No. 122 | Seq Id No. 123 |
| CLCN7 | TGATCTCCACGTTCACCCTGA | TCTCCGAGTCAAACCTTCCGA |
| | Seq Id No. 124 | Seq Id No. 125 |
| CTSA | CAGGCTTTGGTCTTCTCTCCA | TCACGCATTCCAGGTCTTTG |
| | Seq Id No. 126 | Seq Id No. 127 |
| CTSB | AGTGGAGAATGGCACACCCTA | AAGAAGCCATTGTCACCCCA |
| | Seq Id No. 128 | Seq Id No. 129 |
| CTSD | AACTGCTGGACATCGCTTGCT | CATTCTTCACGTAGGTGCTGGA |
| | Seq Id No. 130 | Seq Id No. 131 |
| CTSF | ACAGAGGAGGAGTTCCGCACTA | GCTTGCTTCATCTTGTTGCCA |
| | Seq Id No. 132 | Seq Id No. 133 |
| GALNS | TTGTCGGCAAGTGGCATCT | CCAAACCACTCATCAAATCCG |
| | Seq Id No. 134 | Seq Id No. 135 |
| GBA | TGGGTACCCGGATGATGTTA | AGATGCTGCTGCTCTCAACA |
| | Seq Id No. 136 | Seq Id No. 137 |
| GLA | AGCCAGATTCCTGCATCAGTG | ATAACCTGCATCCTTCCAGCC |
| | Seq Id No. 138 | Seq Id No. 139 |
| GNS | CCCATTTTGAGAGGTGCCAGT | TGACGTTACGGCCTTCTCCTT |
| | Seq Id No. 140 | Seq Id No. 141 |
| HEXA | CAACCAACACATTCTTCTCCA | CGCTATCGTGACCTGCTTTT |
| | Seq Id No. 142 | Seq Id No. 143 |
| LAMP1 | ACGTTACAGCGTCCAGCTCAT | TCTTTGGAGCTCGCATTGG |
| | Seq Id No. 144 | Seq Id No. 145 |
| MCOLN1 | TTGCTCTCTGCCAGCGGTACTA | GCAGTCAGTAACCACCATCGGA |
| | Seq Id No. 146 | Seq Id No. 147 |
| NAGLU | CAGAAGGAAGGAGCAGGAGT | ATGTTCCCGAGGCTGTCAC |
| | Seq Id No. 148 | Seq Id No. 149 |
| NEU1 | CAGCACATCCAGAGTTCCGAGT | TGTCTCTTTCCGCCATGAGGT |
| | Seq Id No. 150 | Seq Id No. 151 |
| PSAP | GCCAACAGTGAAATCCCTTCC | TCAGTGGCATTGTCCTTCAGC |
| | Seq Id No. 152 | Seq Id No. 153 |
| SCPEP1 | GATCTCCCCTGTTGATTCGGT | AGCCCCTTATTTACGGCATTC |
| | Seq Id No. 154 | Seq Id No. 155 |
| SGSH | TGACCGGCCTTTCTTCCTCTA | GCTCTCTCCGTTGCCAAACTT |
| | Seq Id No. 156 | Seq Id No. 157 |
| TMEM55B | GTTCGATGCCCCTGTAACTGTC | CCCAGGTTGATGATTCTTTTGC |
| | Seq Id No. 158 | Seq Id No. 159 |
| TPP1 | GATCCCAGCTCTCCTCAATACG | GCCATTTTTGCACCGTGTG |
| | Seq Id No. 160 | Seq Id No. 161 |
| GAPDH | TGCACCACCAACTGCTTAGC | GGCATGGACTGTGGTCATGAG |
| | Seq Id No. 162 | Seq Id No. 163 |
| HPRT1 | TGACACTGGCAAAACAATGCA | GGTCCTTTTCACCAGCAAGCT |
| | Seq Id No. 164 | Seq Id No. 165 |
| ARPP-19 | AGGAAACGGTTGCAGAAAGG | GTCTTGCGGAGTGGGAATGT |
| | Seq Id No. 166 | Seq Id No. 167 |
| C6orf211 | ACTCACCGTGGTTGTTGGTAGA | TCGATTGGTGGACTCTGGATAA |
| | Seq Id No. 168 | Seq Id No. 169 |
| FBXO11 | GTGATGGACGAGGCCTTATTG | TGCACATAAATCCCACCATGC |
| | Seq Id No. 170 | Seq Id No. 171 |
| HOXA9 | CCCCCATCGATCCCAATAA | CCCTGGTGAGGTACATGTTGAA |
| | Seq Id No. 172 | Seq Id No. 173 |
| KPNA2 | TCCAAGCTACTCAAGCTGCCAG | CCAGCCCGGATTATGTTGTCT |
| | Seq Id No. 174 | Seq Id No. 175 |
| MTDH | CCTCTAAAACCCGTCCAAAACA | TCGGTAGAAGTAGCAGGTGGAA |
| | Seq Id No. 176 | Seq Id No. 177 |
| MTX2 | TGCTGTTGACTGCAGAGCTGT | CCTAGCATGAGTGATCTCCCCT |
| | Seq Id No. 178 | Seq Id No. 179 |
| ONECUT2 | ATGTGGAAGTGGCTTCAGGAG | GGGACTTCTTCTGGGAATTGT |
| | Seq Id No. 180 | Seq Id No. 181 |
| STAT3 | GTCAGGTTGCTGGTCAAATTCC | CAACGTCCCCAGAGTCTTTGTC |
| | Seq Id No. 182 | Seq Id No. 183 |

ChIP assay

| | | |
|---|---|---|
| ATP6V1H | TCGGGAATCCAGTTGTCCG | GCCGCACAGGTAGAAGGAA |
| | Seq Id No. 184 | Seq Id No. 185 |
| CLCN7 | CGTTGCAGGTCACATGGTC | GGCTGCCCCCGTGTTTGT |
| | Seq Id No. 186 | Seq Id No. 187 |
| CTSA | CCGTAGGGACCAAAGAAGG | TGGAAGTCATGTGTACGAGTCA |
| | Seq Id No. 188 | Seq Id No. 189 |
| CTSD | GCGTCATCCCGGCTATAAG | TGAGGCTTCACCTGACGAG |
| | Seq Id No. 190 | Seq Id No. 191 |
| CTSF | AAGCACGTGATAGAGGTCAGTG | CCTGCGCGTTCTCTTGTT |
| | Seq Id No. 192 | Seq Id No. 193 |
| GBA | TGTAACAGATGAGAGGAAGC | ACACAGGAAGTGAGGCAATC |
| | Seq Id No. 194 | Seq Id No. 195 |
| GLA | TAGCGAGACGGTAGACGAC | ACCCGCCCTATTTCCATAC |
| | Seq Id No. 196 | Seq Id No. 197 |
| GNS | ATCGCGCCTAGGGAGAAA | AATAAAAAGCCGTGCCTTGA |
| | Seq Id No. 198 | Seq Id No. 199 |
| HEXA | GTGAAAGGGCAGGGTGTG | CGAATCACGTGACCAGAGG |
| | Seq Id No. 200 | Seq Id No. 201 |
| NEU1 | CTTCGAGATGCTGCGTGAT | TCCCGGACTCTAATTGGTCTT |
| | Seq Id No. 202 | Seq Id No. 203 |
| MCOLN1 | AGGGGCTCTGGGCTACC | GCCCGCCGCTGTCACTG |
| | Seq Id No. 204 | Seq Id No. 205 |
| PSAP | TTGGGGCAGGGCAGATTTAT | CAGGAGGAAGAGGGCGTACA |
| | Seq Id No. 206 | Seq Id No. 207 |
| SCPEP1 | CCGTCCGCCTCCGTCAC | GGCAGCAGCAGCAACCAC |
| | Seq Id No. 208 | Seq Id No. 209 |
| TMEM55B | TCCCAATAGCTTGCAGAACC | TGTCACATGACCTGCCAGA |
| | Seq Id No. 210 | Seq Id No. 211 |
| TPP1 | AGAGGGGTAGTGGTGGTGGAA | CAGGCTTGGAGTCCCATTCT |
| | Seq Id No. 212 | Seq Id No. 213 |
| PSAP(int) | CACAGGCACCCACACAAA | ACGCAGCTGGTCAGCAAT |
| | Seq Id No. 214 | Seq Id No. 215 |
| GNS(int) | ACAAGGAATGGAAACAAAGATACC | GATGCGTCTTCCCTTTTTCC |
| | Seq Id No. 216 | Seq Id No. 217 |
| ACTB | ATGCAGCGATCAGTGGCGT | TCCAGCTTCTTGTCACCACCTC |
| | Seq Id No. 218 | Seq Id No. 219 |
| APRT | GCCTTGACTCGCACTTTTGT | TAGGCGCCATCGATTTTAAG |
| | Seq Id No. 220 | Seq Id No. 221 |
| HPRT | GCCACAGGTAGTGCAAGGTCTT | TTCATGGCGGCCGTAAAC |
| | Seq Id No. 222 | Seq Id No. 223 |
| TXNDC4 | CCTCTCACACCCTCACTTCC | TCTAGATGACGGACGACGTG |
| | Seq Id No. 224 | Seq Id No. 225 |
| WIF1 | GCCAGCTTTGCCAGTCTTAC | CGAGTCGCGCAAGAAGAT |
| | Seq Id No. 226 | Seq Id No. 227 |

TABLE 6
Positional weight matrix (PWM) describing CLEAR sequences
(CLEAR PWM).

| | 1 | 2 | 3 | 4 | 5 | 6 | 7 | 8 | 9 | 10 |
|---|---|---|---|---|---|---|---|---|---|---|
| A | 15 | 1 | 0 | 92 | 6 | 2 | 0 | 0 | 79 | 5 |
| C | 19 | 9 | 94 | 0 | 74 | 12 | 2 | 0 | 5 | 55 |
| G | 55 | 5 | 0 | 2 | 12 | 74 | 0 | 94 | 9 | 19 |
| T | 5 | 79 | 0 | 0 | 2 | 6 | 92 | 0 | 1 | 15 |

### CITED PRIOR ART DOCUMENTS

1. P. Saftig, Lysosomes, Medical Intelligence Unit (Landes Bioscience/Eureka.com, Springer Science+Business Media Inc., Georgetown, New-York, 2003).
2. J. Reimand, M. Kull, H. Peterson, J. Hansen, J. Vilo, Nucleic Acids Res 35, W193 (July, 2007).
3. T. Lubke, P. Lobel, D. E. Sleat, Biochim Biophys Acta (Oct. 15, 2008).
4. M. E. Massari, C. Murre, Mol Cell Bio/20, 429 (January, 2000).
5. N. A. Meadows et al., J Biol Chem 282, 1891 (Jan. 19, 2007).
6. E. Steingrimsson, N. G. Copeland, N. A. Jenkins, Annu Rev Genet 38, 365 (2004).
7. V. A. Gennarino et al., Genome Res 9, 481 (March, 2009).
8. Y. Sancak et al., Science 320, 1496 (Jun. 13, 2008).
9. C. Liang et al., Nat Cell Biol 10, 776 (July, 2008).
10. Y. Shin, J. Klucken, C. Patterson, B. T. Hyman, P. J. McLean, J Biol Chem 280, 23727 (Jun. 24, 2005).
11. S. Kornfeld, W. S. Sly, in The Metabolic and Molecular Basis of Inherited Disease C. R. Scriver, W. S. Sly, A. L. Beaudet, D. Valle, Eds. (McGraw-Hill, New York, 2001).
12. L. E. Karageorgos et al., Exp Cell Res 234, 85 (Jul. 10, 1997).
13. A. Helip-Wooley, J. G. Thoene, Exp Cell Res 292, 89 (2004).
14. E. F. Neufeld, J. Muenzer, in The Metabolic and Molecular Basis of Inherited Disease C. R. Scriver, W. S. Sly, A. L. Beaudet, D. Valle, Eds. (McGraw-Hill, New York, 2001) pp. 3421-3454.
15. A. Ballabio, V. Gieselmann, Biochim Biophys Acta 1793, 684 (April, 2009).
16. J. Muenzer et al., Acta Paediatr Suppl 91, 98 (2002).
17. K. M. Hemsley, J. J. Hopwood, Behav Brain Res 158, 191 (Mar. 30, 2005).
18. C. Settembre et al., Proc Natl Acad Sci USA 104, 4506 (Mar. 13, 2007).
19. S. Sipione et al., Hum Mol Genet 11, 1953 (Aug. 15, 2002).
20. A. M. Cataldo et al., Neuron 14, 671 (March, 1995).
21. A. M. Cataldo, J. L. Barnett, C. Pieroni, R. A. Nixon, J Neurosci 17, 6142 (Aug. 15, 1997).
22. J. Bendiske, B. A. Bahr, J Neuropathol Exp Neurol 62, 451 (May, 2003).
23. U. S. Ladror, S. W. Snyder, G. T. Wang, T. F. Holzman, G. A. Krafft, J Biol Chem 269, 18422 (Jul. 15, 1994).
24. L. Qiao et al., Mol Brain 1, 17 (2008).
25. W. J. Lukiw, Neuroreport 18, 297 (Feb. 12, 2007).
26. W. J. Lukiw, A. I. Pogue, J Inorg Biochem 101, 1265 (September, 2007).
27. R. Saba, C. D. Goodman, R. L. Huzarewich, C. Robertson, S. A. Booth, PLoS ONE 3, e3652 (2008).
28. M. Thomas-Chollier et al., Nucleic Acids Res 36, W119 (Jul. 1, 2008).
29. V. A. Gennarino et al., Genome Res 9, 481 (March, 2009).
30. J. Reimand, M. Kull, H. Peterson, J. Hansen, J. Vilo, Nucleic Acids Res 35, W193 (July, 2007).
31. J. Muenzer et al., Acta Paediatr Suppl 91, 98 (2002).
32. K. M. Hemsley, J. J. Hopwood, Behav Brain Res 158, 191 (Mar. 30, 2005).
33. C. Settembre et al., Proc Natl Acad Sci USA 104, 4506 (Mar. 13, 2007).
34. M. A. Denti, A. Rosa, O. Sthandier, F. G. De Angelis, I. Bozzoni, Mol Ther 10, 191 (July, 2004).
35. A. Subramanian et al., Proc Natl Acad Sci USA 102, 15545 (Oct. 25, 2005).
36. S. Sipione et al., Hum Mol Genet 11, 1953 (Aug. 15, 2002).

1. A molecule being able to enhance the cellular degradative pathways to prevent or antagonize the accumulation of toxic compounds in a cell, characterized by:
a) acting either directly or indirectly on a CLEAR element to enhance the expression of at least a gene involved in cellular degradative pathways, said CLEAR element comprising at least one repeat of a nucleotide sequence having Seq Id No. 110 as consensus sequence; and
b) belonging to the group of: the TFEB protein, synthetic or biotechnological functional derivative thereof, peptide fragments thereof, chimeric molecules comprising the TFEB protein, synthetic or biotechnological functional derivative thereof; modulator of the TFEB protein activity and/or expression level.

2. The molecule according to claim 1 wherein the CLEAR element comprises at least one repeat of a nucleotide sequence having Seq Id No. 111 as consensus sequence.

3. The molecule according to claim 1 wherein the CLEAR element comprises at least one repeat of a nucleotide sequence selected from the group from Seq Id No. 1 to Seq Id No. 109.

4. The molecule according to claim 3 wherein the CLEAR element comprises at least one repeat of a nucleotide sequence selected from the group consisting of: Seq Id No. 3, Seq Id No. 9, Seq Id No. 13, Seq Id No. 26, Seq Id No. 28, Seq Id No. 30, Seq Id No. 32, Seq Id No. 34, Seq Id No. 36, Seq Id No. 47, Seq Id No. 50, Seq Id No. 53, Seq Id No. 59, Seq Id No. 62, Seq Id No. 77, Seq Id No. 78, Seq Id No. 84, Seq Id No. 85, Seq Id No. 88, Seq Id No. 92, Seq Id No. 94, Seq Id No. 95, Seq Id No. 98, and Seq Id No. 108.

5. The molecule according to claim 1 wherein the chimeric molecule comprises the TFEB protein and a nuclear localization signal (NLS).

6. The molecule according to claim 1 wherein the modulator of the TFEB protein is a microRNA or a microRNA inhibitor.

7. The molecule according to claim 6 wherein the microRNA is miR-128 or a miR-128 inhibitor.

8. The molecule according to claim 1 wherein said gene involved in degradative pathways is a gene expressing a lysosomal protein.

9. (canceled)

10. The molecule according to claim 9 for neurodegenerative disorders' treatments.

11. The molecule according to claim 10 wherein the neurodegenerative disorder belongs to the group of Alzheimer, Parkinson and Huntington diseases.

12. The molecule according to claim 9 for lysosomal storage disorders' treatments.

13. The molecule according to claim 12 wherein the lysosomal storage disorders' belongs to the group of Pompe disease and Multiple Sulfatase Deficiency (MSD).

14. A nucleic acid containing a sequence encoding for the molecule according to claim 1.

15. A vector comprising under appropriate regulative sequence the nucleic acid according to claim 14.

16. The vector according claim 15 for gene therapy.
